(19) United States
(12) Patent Application Publication
(10) Pub. No.: US 2026/0209301 A1
(43) Pub. Date: Jul. 23, 2026

(54) Galanin-2 Receptor Agonists

(71) Applicant: LIFEARC, London (GB)

(72) Inventors: Jeffrey Clifford Jerman, Stevenage (GB); Paul Wright, Stevenage (GB); David Wynick, Bristol (GB)

(21) Appl. No.: 19/141,693

(22) PCT Filed: Dec. 22, 2022
(86) PCT No.: PCT/EP2022/087462

§ 371 Date: Jun. 20, 2025

(51) Int. Cl. C07K 14/575; A61K 38/00; A61P 25/04

(52) U.S. Cl. CPC C07K 14/575; A61P 25/04; A61K 38/00; C07K 2319/30

(57) ABSTRACT

The invention related to a chimeric protein comprising (i) a galanin fragment consisting of residues 2-13 of galanin (Gal2-13) or residues 2-13 of galanin (Gal2-12) or a variant thereof, and (ii) an FC region and well as pharmaceutical composition comprising these proteins. These proteins are suitable for use in the treatment of pain.

### FIELD

The present invention relates to Galanin-2 Receptor (GalR2) agonists and their use in the treatment of pain.

### BACKGROUND

Pain is normally associated with injury or damage and causes guarding and immobilisation of the affected area, resulting in protection and the promotion of rapid healing, albeit triggering an unpleasant sensory and emotional experience. Chronic nerve damage or injury in humans (also known as neuropathy) induces an alteration of many of the properties of the primary sensory neurons in the dorsal root ganglion (DRG), and their central connections, leading to the development of spontaneous pain, allodynia (the perception of pain from a normally innocuous stimulus), hyperalgesia (an exaggerated response to any given pain stimulus) and expansion of the receptive pain field (a much larger area of the body “hurts” in relation to a relatively focused stimulus). These functional changes result in the development of chronic neuropathic pain states which are frequently resistant to conventional analgesic therapies.

Galanin was originally isolated from porcine intestine in 19831 and the cDNA subsequently cloned in 1987 from a rat anterior pituitary library². In the adult rodent, primate and human, galanin is expressed at low levels in <5% of intact DRG neurons, which are predominantly small diameter C-fibre nociceptors³⁻⁵ Higher levels of the peptide are also detected in the primary afferent terminals of the spinal cord (lamina II), the dorsal horn inter-neurons⁶, and in a number of brain regions that are thought to be important for pain processing^{7,8}. After the various nerve injury models of Neuropathic Pain (NP) described below, galanin levels in the DRG rise by 5-10 fold in the rodent, primate and human, and the peptide is abundantly expressed in ~20-30% of sensory neurons⁹⁻¹¹. The rise in expression in the dorsal horn following axotomy is modest compared to the marked elevation in the DRG¹², and may reflect in part, an increase in anterograde transport of galanin from the cell body to the site of injury¹³, similar to axotomised sympathetic neurons¹⁴.

Behavioural and electrophysiological studies in intact rats have demonstrated that intrathecal administration of galanin has facilitatory effects on nociception (the neuronal signalling that underlies the sensation of pain) at low doses^{15; 16} with a marked inhibition at higher doses^{17; 18}. After nerve injury, when the endogenous levels of galanin in the DRG are high, there is good agreement in the literature that the neuropeptide inhibits synaptic strength in a number of spinal cord preparations from terminally anaesthetised animals¹⁹. To further define the role played by galanin in modulating NP in an awake and freely moving animal, two novel transgenic lines have been generated and characterised. One line inducibly over-expressed galanin (by ~4-fold) in the DRG after nerve injury²⁰ while the other constitutively and ectopically (by ~5-fold) over-expressed galanin in both the intact DRG and after nerve-injury²¹. The rat spared nerve injury (SNI) model of NP was then modified and applied to wild-type mice, demonstrating that allodynia develops within 24 hours of the lesion in all mouse strains thus far tested and lasts for at least 6 months. Both of the novel galanin over-expressing lines demonstrate a marked reduction in mechanical allodynia in the SNI model²¹ that continues for at least 3 months, demonstrating that tachyphylaxis does not occur in the face of sustained elevated levels of galanin. Subsequently, a similar decrease in NP behaviour has been shown in transgenic mice that ectopically over-express galanin in the DRG under the control of the dopamine beta-hydroxylase promoter²². Most recently, a novel transgenic line using the binary transgenic tet-off system, has been shown to over-express galanin in the DRG in a suppressible manner²⁶. Phenotypic analysis revealed markedly attenuated allodynia when galanin is over-expressed and an increase in allodynia following galanin suppression. This novel transgenic line demonstrates that whether galanin expression is increased at the time of nerve injury or only after allodynia is established, the neuropeptide is able to reduce NP behaviour²³. In summary, the above animal data, using a range of pharmacological and genetic tools and various NP models, supports an inhibitory role for galanin in pain processing in the intact animal and, in particular, after nerve injury.

The physiological effects of galanin are mediated by the activation of one or more of three G-protein coupled galanin receptor subtypes, designated GalR1, GalR2 and GalR3. All three receptors couple to G_{i/o} and inhibit adenylyl cyclase^{24, 25}, and in addition GalR2 also signals via G_{q/11} to activate phospholipase C (PLC) and protein kinase C (PKC)²⁶. Studies using in-situ hybridization have shown that GalR1 and GalR2 mRNAs are expressed by 51% and 83% of adult rat DRG neurons respectively²⁷ and the levels of both sub-types decrease after axotomy^{28; 29}. In contrast, no change in the expression of either subtype was observed in the dorsal horn of the spinal cord after axotomy³⁰. Most recently, specific and highly sensitive semi-quantitative RT-PCR (Taqman) assays have been used to measure levels of mouse GalR1 and GalR2 in the DRG one week after axotomy and demonstrate that expression fell by 37% and 28%, respectively in wild-type (WT) animals³¹. Expression of GalR3 in the rat and mouse spinal cord and DRG is very low as determined by RT-PCR^{31, 32} and undetectable using in situ hybridisation³³.

Analyses of the nociceptive phenotypes of the existing galanin receptor knockout (KO) animals have been largely uninformative with respect to NP. GalR1-KO mice have no change in the degree of allodynia and only minor deficits in its rate of recovery in two different models of NP^{34, 35}. The recently described developmental deficits that occur in a subset of nociceptors (pain sensing neurons) in the DRG of GalR2-KO mice shortly after birth³¹ precludes an analysis of the role played by GalR2 in NP in the adult animal, though still allows us to use these mutant mice as a source of tissue for various GalR2-dependent functional assays. GalR3-KO animals have been described⁶⁶ though no significant NP phenotype has been published.

To date, few high-affinity galanin receptor ligands have been described. Gal2-11 (also known as AR-M1896) has equal affinity for GalR2 and GalR3 and does not activate GalR1^{36; 37}. Attempts by a number of groups to generate GalR1- or GalR2-specific peptide agonists have been largely unsuccessful and only “receptor-preferring” agonists have thus far been described^{38; 39}. Further, galanin and Gal2-11 both have half-lives of <10 minutes in plasma^{40; 41} precluding their systemic administration. Intrathecal infusion of galanin or Gal2-11 in a rat NP model demonstrates that the central effects of galanin are likely to be mediated by activation of GalR1 in the spinal cord³⁶. Recently, activation of either GalR1 or GalR2 (using either a cocktail of a pan-GalR1/2 agonist in combination with a GalR2 antagonist or using AR-M1896) in the dorsal horn of the spinal cord has been shown to alter the electrophysiological properties of inhibitory neurons compatible with an anti-nociceptive role for both receptor subtypes⁴². This conclusion is further strengthened by the demonstration that GalR2 activation directly inhibits calcium currents in cultured DRG neurons⁴³.

These reports provide a mechanism of action by which GalR2 activation in the DRG and/or dorsal horn may reduce NP. These findings are also consistent with previous work demonstrating that modulation of calcium channels by gabapentinoids, ziconotide and opioids all reduce NP in patients. Most recently, electrophysiology has been used to show that exogenous galanin altered the responses of mechano-nociceptive C-fibre afferents in a dose-dependent manner in both naive and nerve injured animals, with low concentrations facilitating and high concentrations markedly inhibiting mechano-nociceptor activity⁴⁴. Further, use of the galanin fragment Gal2-11 confirmed that the effects of galanin were mediated by activation of GalR2. The inhibitory effects of peripheral GalR2 activation were further supported by the demonstration that after a nerve injury model of NP, mechano-sensitive nociceptors in galanin over-expressing transgenic mice had significantly higher thresholds than in wild type animals, associated with a marked reduction in spontaneous neuronal firing and C-fiber barrage into the spinal cord⁴⁴.

Pharmacological study of galanin and its receptors has largely comprised use of native peptide truncates and conjugates. Full length galanin shows high (single digit nM) affinity for GalR1, GalR2 and GalR3. Rat, human and porcine galanin show a similarly high affinity for the human receptor⁴⁸. Residues 1-15 are fully conserved across all species and this fragment binds both GalR1 and GalR2 with a high affinity⁴⁹. Studies have shown that Gal1-15, but not Gal1-29, elicits a functional response in dorsal hippocampus regions of the brain⁵⁰ and this fragment preferentially activates GalR1-GalR2 hetero-complexes⁵¹. Gal1-10 is the shortest fragment shown to retain binding activity with further truncations leading to a total loss of affinity⁵². Residues 17-29 show the highest sequence divergence across species and this fragment was unable to displace radiolabeled galanin in binding studies or show a functional response when tested in vivo⁵³. This has led to speculation that the primary role of this portion of galanin is to protect the N-terminal region (e.g. residues 1 to 15) from proteolytic degradation⁵⁴.

Removal of the N-terminal glycine of galanin gives rise to selectivity toward GalR2 over GalR155 and further truncation of the peptide to the 10 residue fragment Gal2-11, completely abolishes GalR1 activity (and affinity) whilst retaining GalR2 activity⁵⁶. Numerous chimeric galanin agonists have also been described⁵⁷, fusing fragments of galanin with spantide, Neuropeptide Y, mastoparan and bradykinin. M617, comprised of residues 1-13 of galanin, fused to residues 2-9 of bradykinin, with a glutamine at position 14, shows ~25 fold selectivity for GalR1 compared to GalR258. M1145, which shows 90-fold selectivity for GalR2 is comprised of galanin residues 1-13 and residues of GALP. With greater focus on therapeutic intervention a further subset of galanin analogs have been generated aimed at improving pharmacokinetic properties. Nax505-5 (Gal-B2), is a 17 residue galanin fragment containing a lipo-amino acid and additional C-terminal lysine residues and showed increased serum stability, blood brain barrier penetration⁵⁹ and efficacy in seizure and epilepsy models. Nax409-9 (also referred to as GalR2-dPEG24), a monodisperse oligo-ethylene glycol-containing galanin analogue encompassing galanin residues 2-13⁶⁰, was generated as a peripherally restricted GalR2 preferring analogue. This molecule showed efficacy in neuropathic pain models, though with a biological effect of <4 hours. There are two described non-peptide galanin analogues, galnon⁶¹ and galmic⁶², both of which are low affinity and not receptor-subtype specific.

Although the above studies suggest that GalR2 agonism may affect neuropathic pain in model systems, there are no reports of long-lasting GalR2 agonists with the necessary specificity to provide an effective pain treatment.

### SUMMARY

The present inventors have unexpectedly discovered that chimeric proteins comprising a galanin (Gal) fragment consisting of Gal residues 2-13 or variants thereof and the fragment crystallisable (FC) region of an immunoglobulin molecule display long-lasting and highly selective GalR2 agonism in vivo compared to chimeric proteins containing the FC region in combination with other Gal fragments.

### A First Aspect of the Invention Provides a Chimeric Protein Comprising;

(i) a galanin fragment consisting of residues 2-13 of galanin (Gal2-13) or of 2-12 of galanin (Gal2-12) or a variant thereof and
(ii) an FC region.

### Preferably the Chimeric Protein Comprises:

(i) a galanin fragment consisting of residues 2-13 of galanin (Gal2-13) or a variant thereof and
(ii) an FC region.

Preferably, the galanin fragment of the chimeric protein of the first aspect consists of SEQ ID NO: 9 or SEQ ID NO: 10, most preferably SEQ ID NO: 4.

A second aspect of the invention provides a nucleic acid encoding a chimeric protein of the first aspect.

A third aspect of the invention provides a vector comprising a nucleic acid of the second aspect.

A fourth aspect of the invention provides a recombinant cell comprising a vector of the third aspect.

A fifth aspect of the invention provides a pharmaceutical composition comprising a chimeric protein of the first aspect and a pharmaceutically acceptable excipient.

A sixth aspect of the invention provides a method of producing a pharmaceutical composition comprising admixing a chimeric protein of the first aspect with a pharmaceutically acceptable excipient.

A seventh aspect of the invention provides a chimeric protein of the first aspect for use in the treatment of the human or animal body.

An eighth aspect of the invention provides a method of treatment of pain comprising administering a therapeutically effective amount of a chimeric protein of the first aspect or a pharmaceutical composition of the fifth aspect.

A ninth aspect of the invention provides a chimeric protein of the first aspect or a pharmaceutical composition of the fifth aspect for use in the treatment of pain.

A tenth aspect of the invention provides the use of a chimeric protein of the first aspect or a pharmaceutical composition of the fifth aspect in the manufacture of a medicament for the treatment of pain.

Pain in accordance with the eighth, ninth and tenth aspects may be neuropathic pain (NP) or inflammatory pain.

Other aspects and embodiments of the invention are described in more detail below.

### BRIEF DESCRIPTION OF THE FIGURES

FIG. 1 shows the relative potencies of Galanin and FC fusions in GalR2 functional calcium imaging assays.

FIG. 2 shows in vitro profiling using the β-arrestin assay of FC fusions focusing on amino acid positions 12 and 13.

FIG. 3 shows binding affinity of Gal-FC fusions at human GalR2.

FIG. 4 shows the activity of Gal-FC fusions at the three known human galanin receptor subtypes.

FIG. 5 shows a summary of the effect of Ala substitution on the potency and selectivity of Gal2-13 for GalR2 vs GalR1.

FIG. 6 shows the effect of Gal2-30FC on withdrawal threshold in the mouse CCI model of neuropathic pain.

FIG. 7 shows the effect of Gal2-13FC at doses ranging 1-30 mg/kg on withdrawal threshold in the mouse CCI model of neuropathic pain.

FIG. 8 shows the effect of Gal2-13FC at doses ranging 0.1-1 mg/kg on withdrawal threshold in the mouse CCI model of neuropathic pain.

FIG. 9 shows the effect of Gal2-13FC on weight bearing in the mouse CFA model of inflammatory pain.

FIG. 10 shows the longevity of effect of 1 mg/kg Gal2-13FC on withdrawal threshold in the mouse CCI model of neuropathic pain.

FIG. 11 shows the effect of Gal2-13FC at doses ranging 0.01-0.1 mg/kg on withdrawal threshold in the mouse CCI model of neuropathic pain.

FIG. 12 shows the effect of Gal2-13FC at doses ranging 0.1-3 mg/kg on withdrawal threshold in the mouse chemotherapy-induced model of neuropathic pain.

FIG. 13 shows the effect of repeat administration of Gal2-13FC on withdrawal threshold in the mouse CCI model of neuropathic pain.

### DETAILED DESCRIPTION

This invention relates to a chimeric protein that comprises a fragment of galanin (Gal) (“warhead”) linked to an FC region. The fragment consists of Gal residues 2-13 or 2-12 or a variant thereof. Unlike Gal2-11, these chimeric proteins are highly selective agonists of peripheral GalR2 and have a long duration of effect in vivo. In addition, the chimeric protein is restricted to the peripheral nervous system (i.e. it does not cross the blood brain barrier) and may thus display reduced CNS-mediated side effects, such as sedation or addiction, compared to other analgesics. A chimeric protein described herein may be useful in the treatment of pain, for example neuropathic or inflammatory pain.

Human galanin (Gene ID 51083) is a 30mer neuropeptide that has the reference amino acid sequence of NCBI database entry AAB20740.1. Mature Gal corresponds to residues 33-62 of the Gal pre-pro-peptide, which has the reference amino acid of NP_057057.2 and may be encoded by the reference nucleotide sequence of NM_015973.4.

The Gal fragment of the chimeric protein specifically binds to the Galanin receptor 2 (GalR2). The Gal fragment may bind preferentially or selectively to GalR2 over the Galanin receptor 1 (GalR1) and the Galanin receptor 3 (GalR3). Human Galanin receptor 2 (GalR2 Gene ID 8811) may have the reference amino acid of database entry NP_003848.1 and may be encoded by the reference nucleotide sequence of database entry NM_003857.3.

Preferably the Gal fragment of the chimeric protein consists of residues 2 to 13 of galanin, preferably human galanin or may be a variant thereof. For example, the Gal fragment may consist of the amino acid sequence of WTX₁NSA X₂YLLGX₃ where X₁, X₂ and X₃ are independently any amino acid (SEQ ID NO: 9).

Preferably, X₁ is independently L or A, X₂ is independently G or A, and X₃ is independently P or A. For example, the Gal fragment may consist of SEQ ID NO: 10.

In some preferred embodiments, the Gal fragment may consist of the amino acid sequence of WTLNSAGYLLGP (SEQ ID NO: 4) or may be a variant thereof.

In one embodiment, the Gal fragment may consist of the amino acid sequence of WTLNSAGYLLG (SEQ ID NO: 49) or may be a variant thereof.

The Gal fragment is preferably located at the N terminus of the chimeric protein.

The chimeric protein does not comprise the full length of the galanin, i.e. the chimeric protein does not comprise the sequence GWTLNSAGYLLGPHAVGNHRSFSDKNGLTS (SEQ ID NO: 15), GWTLNSAGYLLGPHAIDNHRSFHDKYGLA (SEQ ID NO: 16) or N-terminus truncated galanin, e.g. does not comprise the sequence of WTLNSAGYLLGPHAVGNHRSFSDKNGLTS (SEQ ID NO: 57) or WTLNSAGYLLGPHAIDNHRSFHDKYGLA (SEQ ID NO: 17).

By “consist of residues 2 to 13 of galanin” or similar, it is meant that the chimeric protein only contains of residues 2 to 13 of Gal, e.g. the Gal fragment portion of the chimeric protein has a length of 12 amino acids. By “consist of residues 2 to 12 of galanin” or similar, it is meant that the chimeric protein only contains residues 2 to 12 of Gal, e.g. the Gal fragment portion of the chimeric protein has a length of 11 amino acids. For example, the chimeric protein does not comprise the sequence WTLNSAGYLLGPHA (SEQ ID NO: 58), WTLNSAGYLLGPH (SEQ ID NO: 59) or variants thereof.

An antibody fragment crystallisable region (FC region) may comprise the second and third constant domains (CH2 and CH3) of an immunoglobulin heavy chain. Suitable immunoglobulin FC regions for use in the chimeric proteins described herein are well-known in the art and include an IgG FC region, preferably an IgG1 or IgG4 FC region. In some embodiments, the FC region of a chimeric protein described herein may comprise the amino acid sequence of SEQ ID NO: 5 or may be a variant thereof.

In some embodiments, the FC region may display reduced immunogenicity and/or effector activity (e.g. reduced antibody-dependent cell-mediated cytotoxicity (ADCC), antibody-dependent cellular phagocytosis (ADCP), and/or complement-dependent cytotoxicity (CDC) activity). For example, a chimeric protein may comprise an FC region that has low effector activity, such as an IgG4 FC region; and/or an FC region, such as an IgG1, IgG2 or IgG4 FC region, that comprises one or more amino acid mutations, such as substitutions, deletions or insertions, that reduce or eliminate immunogenicity and/or effector activity. Suitable mutated FC regions are known in the art (see for example WO2004/110472; WO2005/000892; WO2005/113606; Wang et al (2018) Protein Cell 9 (1) 63-73; Saxena et al Front Immunol 2016 7 580; Lo et al; 2017 JBC 292 3900-3908; Jacobsen et al (2017) JBC 292 1865-1875).

The FC region is preferably located at the C terminus of the chimeric protein. The Gal fragment may be connected via a linker or more preferably may be connected to the FC region directly.

Suitable linkers are well-known in the art and include chemical and more preferably peptidyl linkers.

A peptidyl linker may comprise a sequence of amino acid residues, for example, 3-15 amino acid residues, preferably 9 to 15 amino acid residues, more preferably 11 to 13 amino acid residues, even more preferably about 12 amino acid residues. Any linker sequence may be employed. Preferably, the linker sequence is a heterologous sequence and is non-immunogenic. Suitable linker amino acid sequences are well known in the art and may include the amino acid sequence AEAAAKEAAAKA (SEQ ID NO: 6), GGS (SEQ ID NO: 11), GGSGGS (SEQ ID NO: 12), GGSGGSGGSGGS (SEQ ID NO: 13) or PAPAPAPA (SEQ ID NO: 14) or a variant of any of these sequences.

A chimeric protein as described herein may comprise the amino acid sequence of SEQ ID NO: 7 or SEQ ID NO: 8 or may be a variant of one of these sequences. In one embodiment the chimeric protein as described herein may comprise the amino acid sequence of SEQ ID NO: 60 or SEQ ID NO: 25 or may be a variant of these sequence.

A chimeric protein, linker, binding moiety or FC region as described herein that is a variant of a reference sequence, such as a reference sequence described above, may have 1 or more amino acid residues altered relative to the reference sequence. For example, 50 or fewer amino acid residues may be altered relative to the reference sequence, preferably 45 or fewer, 40 or fewer, 30 or fewer, 20 or fewer, 15 or fewer, 10 or fewer, 5 or fewer or 3 or fewer, 2 or 1. For example, a variant described herein may comprise the sequence of a reference sequence with 50 or fewer, 45 or fewer, 40 or fewer, 30 or fewer, 20 or fewer, 15 or fewer, 10 or fewer, 5 or fewer, 3 or fewer, 2 or 1 amino acid residues mutated. For example, a chimeric protein described herein may comprise an amino acid sequence with 50 or fewer, 45 or fewer, 40 or fewer, 30 or fewer, 20 or fewer, 15 or fewer, 10 or fewer, 5 or fewer, 3 or fewer, 2 or 1 amino acid residue altered relative to SEQ ID NO: 7 or SEQ ID NO: 8.

An amino acid residue in the reference sequence may be altered or mutated by insertion, deletion or substitution, preferably substitution for a different amino acid residue. Such alterations may be caused by one or more of addition, insertion, deletion or substitution of one or more nucleotides in the encoding nucleic acid.

A chimeric protein, linker, binding moiety or FC region as described herein that is a variant of a reference sequence may share at least 50% sequence identity with the reference amino acid sequence, at least 55%, at least 60%, at least 65%, at least 70%, at least about 80%, at least 90%, at least 95%, at least 98% or at least 99% sequence identity. For example, a chimeric protein described herein may comprise an amino acid sequence that has at least 50% sequence identity with the reference amino acid sequence, at least 55%, at least 60%, at least 65%, at least 70%, at least about 80%, at least 90%, at least 95%, at least 98% or at least 99% sequence identity with SEQ ID NO: 7 or SEQ ID NO: 8.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm may be used (Nucl. Acids Res. (1997) 25 3389-3402). Sequence identity and similarity may also be determined using Genomequest™ software (Gene-IT, Worcester MA USA). Sequence comparisons are preferably made over the full-length of the relevant sequence described herein

A chimeric protein described herein may further comprise one or more heterologous amino acid sequences additional to the FC domain, Gal fragment and linker. For example, the chimeric protein may further comprise one or more additional domains which improve the stability, pharmacokinetic, targeting, affinity, purification and production properties of the chimeric protein described herein.

Chimeric proteins as described herein may be provided using synthetic or recombinant techniques which are standard in the art.

In some embodiments, the chimeric protein described herein may be produced with an affinity tag, which may, for example, be useful for purification. An affinity tag is a heterologous peptide sequence which forms one member of a specific binding pair. Polypeptides containing the tag may be purified by the binding of the other member of the specific binding pair to the polypeptide, for example in an affinity column. For example, the tag sequence may form an epitope which is bound by an antibody molecule.

Suitable affinity tags include for example, glutathione-S-transferase, (GST), maltose binding domain (MBD), MRGS(H)₆ (SEQ ID NO: 50), DYKDDDDK (FLAG™) (SEQ ID NO: 51), T7—, S-(KETAAAKFERQHMDS) (SEQ ID NO: 52), poly-Arg (R₅₋₆), poly-His (H₂₋₁₀), poly-Cys (C₄) poly-Phe(F₁₁) poly-Asp(D₅₋₁₆), SUMO tag (Invitrogen Champion pET SUMO expression system), Strept-tag II (WSHPQFEK) (SEQ ID NO: 53), c-myc (EQKLISEEDL) (SEQ ID NO: 54), Influenza-HA tag (Murray, P. J. et al (1995) Anal Biochem 229, 170-9), Glu-Glu-Phe tag (Stammers, D. K. et al (1991) FEBS Lett 283, 298-302), Tag. 100 (Qiagen; 12 aa tag derived from mammalian MAP kinase 2), Cruz tag 09™ (MKAEFRRQESDR, Santa Cruz Biotechnology Inc.) (SEQ ID NO: 55) and Cruz tag 22™ (MRDALDRLDRLA, Santa Cruz Biotechnology Inc.) (SEQ ID NO: 56). Known tag sequences are reviewed in Terpe (2003) Appl. Microbiol. Biotechnol. 60 523-533. In preferred embodiments, a poly-His tag such as (H)₅, His-SUMO tag (Invitrogen Champion pET SUMO expression system), or MRGS (H)₆ may be used. The affinity tag sequence may be separated from the chimeric protein described herein after purification, for example, using site-specific proteases.

In some embodiments, the chimeric protein described herein may be coupled to a leader peptide to direct secretion of the chimeric protein from cell into the culture medium as a precursor chimeric protein. A range of suitable leader peptides are known in the art and include SEQ ID NO: 3 or a variant thereof. The leader peptide may be heterologous to the Gal fragment described herein i.e. it may be a non-galanin leader sequence. For example, an α-factor secretion signal or BiP leader sequence may be employed. The leader peptides is located at the N terminus of the precursor chimeric protein. The Gal fragment may be located immediately adjacent the N-terminal leader sequence in the precursor chimeric protein (i.e. the N terminus of the binding moiety is directly linked to the C terminus of the leader sequence). The leader peptide is then removed by post-translational processing after expression of the precursor to generate the chimeric protein.

Chimeric proteins as described herein may be isolated, in the sense of being free from contaminants, such as other polypeptides and/or cellular components.

A chimeric protein as described herein may be a selective agonist of GalR2 in vitro i.e. it may activate GalR2 preferentially or selectively relative to GalR1 and/or GalR3. The in vitro activity of the chimeric protein may be determined by any convenient method. For example, the effect of the chimeric protein on GalR2 may be determined by measuring intracellular calcium mobilization and the effect of the chimeric protein on GalR1 activation may be determined by measuring cyclic AMP (CAMP). The effect of the chimeric protein on both GalR1 and GalR2 may be determined by measuring β-arrestin (BA) recruitment, as described herein. Suitable assays are described in detail below.

The chimeric protein may fail to cross the BBB or penetrate the CNS in vivo and so may fail to activate GalR2 in CNS neurons. The chimeric protein may therefore be a selective agonist of peripheral GalR2 i.e. it may activate GalR2 in peripheral neurons but may show no activation or substantially no activation of GalR2 in CNS neurons. Because it does not activate GalR2 in CNS neurons, a chimeric protein as described herein may not exert a sedative effect or other CNS mediated effects in vivo.

A chimeric protein described herein may exert an analgesic effect in vivo. The analgesic effect may be a peripheral analgesic effect i.e. it may affect peripheral neurons and not CNS neurons. The in vivo activity of the chimeric protein may be determined by any convenient method. For example, the ability of the chimeric protein to reduce, inhibit or reverse mechanical and/or thermal hyperalgesia or allodynia in the mouse Chronic Constriction Injury (CCI) model or complete Freund's adjuvant (CFA) induced hyperalgesia or allodynia in a mouse model (Gould et al Pain. 2000 March; 85 (1-2): 301-3) as may be determined.

A chimeric protein described herein may display biological activity, such as analgesia, in vivo for an extended period, for example for over 24 hours, over 48 hours, over 72 hours or over 96 hours, for example when measured in a mouse model. Suitable models include the CCI model or CFA induced hyperalgesia and or allodynia models, as described above. In some embodiments, a chimeric protein may reduce or reverse mechanical allodynia or hyperalgesia in a model system for at least 48 hours, at least 72 hours or at least 96 hours. For example, a chimeric protein described herein may reduce paw withdrawal in the CCI model or the CFA induced hyperalgesia model relative to a control and/or gabapentin for at least 48 hours, at least 72 hours or at least 96 hours.

The chimeric protein may display biological activity in vivo for longer than a control fusion protein. For example, the chimeric protein may display biological activity for over two-fold longer, over three-fold longer or over four-fold longer than a control fusion protein in which the Gal2-13 fragment is replaced by a Gal2-30 fragment.

Other aspects of the invention provide a nucleic acid encoding a chimeric protein described herein as described above and a vector comprising such a nucleic acid.

Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in mammalian cells. A vector may also comprise sequences, such as origins of replication, promoter regions and selectable markers, which allow for its selection, expression and replication in bacterial hosts such as E. coli.

Vectors may be plasmids, viral e.g. phage, or phagemid, as appropriate. For further details see, for example, Molecular Cloning: a Laboratory Manual: 3rd edition, Russell et al., 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, are described in detail in Current Protocols in Molecular Biology, Ausubel et al. eds. John Wiley & Sons, 1992.

A nucleic acid or vector as described herein may be introduced into a host cell.

Another aspect of the invention provides a recombinant cell comprising a nucleic acid or vector that expresses a chimeric protein as described above.

A range of host cells suitable for the production of recombinant chimeric proteins are known in the art. Suitable host cells may include prokaryotic cells, in particular bacteria such as Escherichia coli and Lactococcus lactis and eukaryotic cells, including mammalian cells such as CHO and CHO-derived cell lines (Lec cells), HeLa, COS, HEK293 and HEK-EBNA cells, amphibian cells such as Xenopus oocytes, insect cells such as Trichoplusia ni, Sf9 and Sf21 and yeast cells, such as Pichia pastoris.

Techniques for the introduction of nucleic acid into cells are well established in the art and any suitable technique may be employed, in accordance with the particular circumstances. For eukaryotic cells, suitable techniques may include calcium phosphate transfection, DEAE-Dextran, electroporation, liposome-mediated transfection and transduction using retrovirus or other virus, e.g. adenovirus, AAV, lentivirus or vaccinia. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well-known in the art.

The introduced nucleic acid may be on an extra-chromosomal vector within the cell or the nucleic acid may be integrated into the genome of the host cell. Integration may be promoted by inclusion of sequences within the nucleic acid or vector which promote recombination with the genome, in accordance with standard techniques.

The introduction may be followed by expression of the nucleic acid to produce the encoded chimeric protein described herein. The host cell may be an in vitro host cell. In some embodiments, host cells (which may include cells actually transformed although more likely the cells will be descendants of the transformed cells) may be cultured in vitro under conditions for expression of the nucleic acid, so that the encoded serpin polypeptide is produced. When an inducible promoter is used, expression may require the activation of the inducible promoter.

The expressed polypeptide comprising or consisting of the chimeric protein may be isolated and/or purified, after production. This may be achieved using any convenient method known in the art. Techniques for the purification of recombinant polypeptides are well known in the art and include, for example HPLC, FPLC or affinity chromatography. In some embodiments, purification may be performed using an affinity tag on the polypeptide as described above.

Another aspect of the invention provides a method of producing a chimeric protein described herein comprising expressing a nucleic acid encoding the chimeric protein in a host cell and optionally isolating and/or purifying the chimeric protein thus produced.

After production, the chimeric protein may be investigated further, for example the pharmacological properties and/or activity may be determined. Methods and means of protein analysis are well-known in the art.

A chimeric protein described herein as described herein may be useful in therapy. For example, the chimeric protein may be administered to an individual for the treatment of pain.

Whilst the chimeric protein may be administered alone, it will usually be administered in the form of a pharmaceutical composition, which may comprise at least one component in addition to the chimeric protein. Thus pharmaceutical compositions may comprise, in addition to the chimeric protein itself, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by bolus, infusion, injection or any other suitable route, as discussed below.

In some embodiments, the chimeric protein may be provided in a lyophilised form for reconstitution prior to administration. For example, a lyophilised chimeric protein may be re-constituted in sterile water and mixed with saline prior to administration to an individual.

For parenteral, for example sub-cutaneous or intra-venous administration, e.g. by injection, the pharmaceutical composition comprising the chimeric protein described herein, nucleic acid or cell may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG). Suitable carriers, excipients, etc. can be found in standard pharmaceutical texts, for example, Remington's Pharmaceutical Sciences, 18th edition, Mack Publishing Company, Easton, Pa., 1990.

Pharmaceutical compositions and formulations may conveniently be presented in unit dosage form and may be prepared by any methods well known in the art of pharmacy. Such methods include the step of bringing into association the chimeric protein described herein with the carrier which constitutes one or more accessory ingredients. In general, the compositions are prepared by uniformly and intimately bringing into association the active compound with liquid carriers or finely divided solid carriers or both, and then if necessary shaping the product.

Preferably a chimeric protein or composition as described herein may be administered by infusion intravenously or subcutaneously.

A pharmaceutical composition comprising a chimeric protein described herein may be administered alone or in combination with other treatments, either simultaneously or sequentially dependent upon the condition to be treated.

A chimeric protein described herein may be used in a method of treatment of the human or animal body, including therapeutic and prophylactic or preventative treatment (e.g. treatment before the onset of a condition in an individual to reduce the risk of the condition occurring in the individual; delay its onset; or reduce its severity after onset). The method of treatment may comprise administering a chimeric protein described herein to an individual in need thereof.

A method of treatment of pain may comprise administering a therapeutically effective amount of the chimeric protein or pharmaceutical composition described herein. Related aspects of the invention provide a chimeric protein or pharmaceutical composition for use in the treatment of pain and the use of a chimeric protein or pharmaceutical composition described herein in the manufacture of a medicament for the treatment of pain.

Pain may include neuropathic pain (NP), for example peripherally or centrally mediated neuropathic pain; inflammatory pain; nociceptive pain; or chronic pain.

In some embodiments, pain that may be treated as described herein may include the symptoms of allodynia; hyperalgesia; spontaneous pain; or expansion of the receptive field. Causes of these pain-related symptoms may include diabetic peripheral neuropathy, arthritic pain, post-herpetic neuralgia, trigeminal neuralgia, post-stroke pain, multiple sclerosis-associated pain, neuropathy-associated pain such as in idiopathic or post-traumatic neuropathy and mononeuritis, HIV-associated neuropathic pain, cancer-associated neuropathic pain, carpal tunnel-associated neuropathic pain; hyperalgesia, for example opioid-induced hyperalgesia, sciatica, spinal cord injury-associated pain, complex regional pain syndrome, fibromyalgia-associated neuropathic pain, lumbar and cervical pain, reflex sympatric dystrophy, phantom pain, phantom limb syndrome, peripheral nerve or spinal cord trauma, entrapment neuropathy, nerve transection including surgery, Lissauer tract section, limb amputation, stump pain, neuroma/tumour compression, arteriovenous malformation, Vitamin B12 deficiency, diabetic neuropathy, alcoholic neuropathy, pain caused by the side effects of anti-cancer and anti-AIDS therapies, pain associated with inflammation or infection of a tooth (toothache), visceral pain, pain caused by chemical burns, pain caused by local or systemic infection, or pain caused by connective tissue disease, such as rheumatoid arthritis, Wallenberg's syndrome, systemic lupus erythematosus, multiple sclerosis, and polyarteritis nodosa.

Pain may, for example be cancer-, surgery-, visceral damage-, headache- or trauma-associated pain.

An individual suitable for treatment as described above may be a mammal, such as a rodent (e.g. a guinea pig, a hamster, a rat, a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-outan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

Administration is normally in a “therapeutically effective amount” or “prophylactically effective amount”, this being sufficient to show benefit to a patient. Such benefit may be at least amelioration of pain in the patient. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners.

A composition may be administered alone or in combination with other treatments, for example treatment with other analgesics, such as paracetamol, non-steroidal anti-inflammatory drugs (NSAIDs), opioids, anti-epileptics such as gabapentin, pregabalin or carbamazepine or anti-depressants such as amitriptyline or duloxetine, either simultaneously or sequentially dependent upon the circumstances of the individual to be treated.

Prescription of treatment, e.g. decisions on dosage etc, is within the responsibility of general practitioners and other medical doctors and may depend on the severity of the symptoms and/or progression of a disease being treated. Appropriate doses of therapeutic polypeptides are well known in the art (Ledermann J. A. et al. (1991) Int. J. Cancer 47:659-664; Bagshawe K. D. et al. (1991) Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of a chimeric protein described herein may be determined by comparing it's in vitro activity and in vivo activity in an animal model. Methods for extrapolation of effective dosages in mice and other test animals to humans are known. The precise dose will depend upon a number of factors, including whether the chimeric protein described herein is for prevention or for treatment, the size and location of the area to be treated, the precise nature of the chimeric protein described herein and the nature of any detectable label or other molecule attached to the chimeric protein described herein.

An initial higher loading dose, followed by one or more lower doses, may be administered. This is a dose for a single treatment of an adult patient, which may be proportionally adjusted for children and infants. Treatments may be repeated at daily, twice-weekly, weekly or monthly intervals, at the discretion of the physician. The treatment schedule for an individual may be dependent on the pharmacokinetic and pharmacodynamic properties of the chimeric protein described herein composition, the route of administration and the nature of the condition being treated.

Treatment may be periodic, and the period between administrations may be about 12 hours or more, 24 hours or more, 36 hours or more, 48 hours or more, 96 hours or more, or one week or more. Chimeric proteins described herein are shown to exert long lasting effects in vivo. In some preferred embodiments, the period between administrations may be one week or more, two weeks or more, or one month or more, for example, administration may be weekly, biweekly or monthly.

Suitable formulations and routes of administration are described above.

Other aspects of the invention provide the use of a chimeric protein described herein as described herein as an analgesic and the use of a chimeric protein described herein as a selective agonist of peripheral GalR2.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.
“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### EXPERIMENTAL

### Materials and Methods

### Cell Culture

CHO cell lines were generated expressing human GalR1 and human GalR2 using the Flp-in system (Life Technologies, USA). GalR1 and GalR2 were cloned into pcDNA5/FRT and transfected into CHO cells using Lipofectamine 2000 (Life Technologies, USA) as per manufactures guidelines. Cells were maintained in F12 Hams, containing 10% (v/v) Fetal bovine serum and 200 μg/ml Hygromycin (all Life Technologies, USA). β-arrestin assays were carried out using PathHunter CHO-K1 GALR2 β-Arrestin Cell Line and PathHunter CHO-K1 GALR1 β-Arrestin Cell Line (DiscoverX, USA). Cells were maintained in DMEM/F12 with 10% FBS, 300 μg/ml Hygromycin and 500 μg/ml Geneticin (all Life Technologies, USA).

### Ligands

Human full length galanin (1-30), rat full length galanin, rat galanin truncate 2-29, galanin truncate 1-15, galanin truncate 2-11, M617, M1145 and Galnon were purchased from Tocris, UK. Ligands were typically re-suspended to 1 mM in water. Galnon was re-suspended in 100% DMSO to final concentration of 10 mM. Nax409-9 was purchased from Cambridge Research Biochemicals, UK. (See Table 1)

### In Vitro Assays

### Calcium Assays

CHO GalR2 cells were plated at 10,000 cells per well in black clear bottom plates (corning, USA) and incubated overnight at 37° C. with 5% CO₂. The following day cell media was removed and replaced with Calcium 4 FLIPR dye (Molecular Devices, USA), re-suspended in HBSS with 2 mM HEPES (Life Technologies, USA and 0.1% BSA. Cells and dye were incubated at 37° C. for 45 minutes, before being allowed to equilibrate to room temperature for ~15 minutes. Ligands were prepared at a 5× concentration and added to cells using the FLIPR (Molecular Devices, USA). Baseline was typically 20 seconds before ligand addition. Fluorescence was measured for 120 seconds, excitation 485 nM, emission 520 nM. For analysis, fluorescence was normalised to pre-addition baseline and peak fluorescence used for all quantification.

### CAMP Assays

The CAMP dynamic 2 kit (Cisbio International) was used as a competitive immunoassay to measure Homogenous Time-Resolved Fluorescence (HTRF). CHO GalR1 cells were plated at 10,000 cells and the following day cell media was removed and replaced with 5 μl of cell assay buffer (Dulbecco's Phosphate Buffered Saline solution (Gibco by Life Technologies), 10 mM 4-(2-hydroxyethyl)-1-piperazineethanesulfonic acid (HEPES, Sigma-Aldrich), 0.1% BSA (Bovine Serum Albumin in DPBS from Sigma-Aldrich) and 0.5 mM of 3-isobutyl-1-methylxanthine (IBMX, Life Technologies) a phosphodiesterase (PDE) inhibitor, to prevent the breakdown of cyclic AMP. Cells were then stimulated with 5 μl of appropriate concentrations of galanin agonists (Tocris, UK) containing forskolin (Cambridge Bioscience, UK) in diluent buffer (DPBS solution, 10 mM HEPES and 0.1% BSA) for 30 minutes at room temperature. Addition of detection reagent involved adding 5 μl d2 conjugated CAMP followed by 5 μl anti-CAMP cryptate conjugate. The two reagents were not pre-mixed; these were both prepared in cell lysis buffer and incubated at room temperature in the dark for one hour. The signal was quantified using the PHERAstar, optimised for HTRF. Forskolin stimulated IC50 and the fluorescence ratio (665 nm/620 nm) were determined by nonlinear regression analysis.

### β-Arrestin Assays

PathHunter CHO-K1 GALR2 β-Arrestin Cells or PathHunter CHO-K1 GALR1 β-Arrestin Cell were typically plated at 5,000 cells per well using Cell plating reagent 2 (DiscoverX, USA) and incubated overnight at 37° C. with 5% CO₂. Ligands were prepared at half log dilutions using cell plating reagent 2. 5 μl of each ligand was added to cells and plates incubated at 37° C. for 90 minutes. DiscoverX PathHunter detection kit (DiscoverX, USA) was added to cells as described my manufacturer's guidelines and plates incubated for 60 minutes at room temperature in the dark. Chemiluminescent signal was measured using the PHERAstar FS (BMG, Germany).

### Radioligand Binding Assays

Cell membrane homogenates (4 μg protein—from CHO cells expressing the GalR2 receptor) were incubated for 120 minutes at 22° C. with 0.05 nM [¹²⁵I] galanin in the absence or presence of test compound in a buffer containing 25 mM Tris-HCl (pH 7.4, 10 mM MgCl₂ and 0.5% BSA. Non-specific binding was determined in the presence of 1 μM porcine galanin. Following incubation, samples were filtered rapidly under vacuum through glass fiber filters (GF/B, Packard) pre-soaked with 0.3% PEI and rinsed several times with ice cold 50 mM Tris-HCl using a 96 sample cell harvester (Unifilter, Packard). The filters are dried then counted for radioactivity in a scintillation counter (Topcount, Packard) using a scintillation cocktail (Microscint 0), Packard). The results are expressed as a percent inhibition of the control radioligand specific binding. The standard reference compound is porcine galanin which is tested in each experiment at several concentrations to obtain a competition curve from which its IC50 is generated.

### GalR3 Reporter Gene Assays

GalR3 activity was measured, alongside GalR1 and GalR2, using a luciferase reporter assay. A reporter plasmid and a receptor plasmid, were transiently transfected into the target cell line. The reporter plasmid contains a serum response element (SRE) or a nuclear factor of activated T-cells response element (NFAT-RE) which is coupled to a luciferase transcript and induces expression of the luciferase upon a specific G-protein (Gai uses SRE; Gaq uses NFAT-RE) activation. The target receptor is constantly expressed after cell transfection. For GALR1 and GALR3 screening, genetically modified HEK293 Gqi cells were transiently transfected with the SRE reporter and the receptor (GALR1 and GALR3) plasmids. For GALR2 screening, HEK293 cells were transiently transfected with the NFAT-RE reporter and the receptor (GALR2) plasmid. The cells were treated with galanin receptor agonists (3 μM-95 μM, half log dilutions) for 5 hours. The luciferase activity was analyzed with the Bright-Glo Luciferase Assay (Promega) in an EnSpire plate reader.

### In Vivo Assays

### Chronic Constriction Injury (CCI) Model of Chronic Pain

The Chronic Constriction Injury (CCI) model of neuropathic pain involves unilateral loose ligation of four ligatures around the left sciatic nerve at mid-thigh level spaced 1 mm apart (Chaplan S R, et al J Neurosci Methods. 1994 July; 53(1):55-63). This procedure results in the development of hyperalgesia, allodynia and spontaneous pain (ectopic discharges) which can be measured using mechanical and thermal behavioural assessments. This model is believed to mimic some of the symptoms and aetiology of neuropathic pain observed in the clinic (Bennett G J, Xie Y K. Pain. 1988 33(1):87-107; Field M J, et al Pain. 1999 November; 83(2):303-11). The mouse CCI model of neuropathic pain differs from that of the rat by virtue of 3 ligatures around the sciatic nerve instead of 4.

Control group: PBS, 10 mL/kg.

Reference group: Pregabalin (15 mg/kg p.o.)

Animals: Male, Naive C57Bl6 mice were age 6 weeks on arrival and were acclimatised to the procedure room in their home cages, with food and water available ad libitum.

Environmental conditions: Animals were housed in standard caging and laboratory conditions in groups of 2-4, with free access to food (5CR4, Purina) and water (except during placement in the test box) on a 12/12 light/dark cycle. Environmental enrichment was supplied from arrival day and changed every three days to help prevent autotomy.

Static mechanical tactile allodynia: Static mechanical (tactile) allodynia was assessed by measurement of withdrawal threshold using calibrated (force; g) von-Frey monofilaments (Touch-Test Sensory Evaluator; Scientific Marketing Associates) applied to the plantar surface of the hindpaw. The animals were placed in individual Perspex boxes on a raised metal mesh for 30-40 min before the test. A series of graduated von Frey hairs (0.07, 0.16, 0.4, 0.6 and 1 g) was applied in sequence with a protocol of 1 sec on 1 sec off repeated 10 times. Each hair was applied perpendicularly to the centre of the ventral surface of the paw until it bent slightly. The force applied to the hind-paw of the animal to induce 5 responses out of 10 trials was recorded as paw withdrawal threshold (PWT).

Baseline PWT was assessed on three consecutive days (Day-2, Day-1 and Day 0) and the mean of the last two readings used as the baseline withdrawal threshold.

On Day 1, neuropathic pain was induced via ligation of the sciatic nerve using three loose ligatures of prolene sutures (Chronic Constriction Injury). Under Isoflurane anaesthesia mixed with oxygen (3:1, 1 L/min) the left hind leg was shaved mid-thigh level and an incision made through the skin using a scalpel. The biceps femoris muscle layer was dissected by making an initial incision using a pair of sharp scissors, which was then widened using a pair of blunt scissors. The common sciatic nerve was exposed using a pair of forceps and 3 loose ligatures of prolene (7-0, Ethicon) were tied around the sciatic nerve with 1 mm spacing between each. The nerve was then returned below the muscle layer and the wound closed using absorbable sutures (Vicryl).

Von Frey assessment of mechanical allodynia was taken on days 19 and 22. Animals were then ranked and randomised (based on a Latin square design) to treatment groups according to the percentage change (compared to pre-surgery baseline) of the mean mechanical withdrawal threshold observed on days 19 and 22. Only those animals showing a PWT percentage change of >60% from pre-surgery baseline were included in the study.

Dosing took place on day 23 with either vehicle, test compound (i.p.) or Pregabalin (15 mg/kg p.o.) at 10 ml/kg. Allodynia was then assessed at intervals thereafter e.g. 2, 4 and 24 hours post dose. von Frey measurement continued until activity disappeared up to day 27.

For studies involving a repeat administration, dosing took place on day 23 with either vehicle, test compound (i.p.) or Pregabalin (15 mg/kg p.o.) at 10 mL/kg. Allodynia was then assessed at intervals thereafter e.g. 4, 24 and 48 hours post dose. Animals were re-dosed at 68, 116, 164, 212, 260, 308 hours, with von Frey measurements conducted 4 hrs after each administration. Allodynia was therefore measured at 72, 120, 168, 216, 264, and 312 hours post initial dose, equating to repeat dosing 2, 3, 4, 5, 6 and 7 times, respectively

Evaluation of study & Statistical analysis: vF withdrawal threshold (g) was assessed by a series of graduated von Frey hairs (0.07, 0.16, 0.4, 0.6 and 1 g) applied in sequence to the ipsilateral hindpaw with a protocol of 1 sec on 1 sec off repeated 10 times. Each hair was applied perpendicularly to the centre of the ventral surface of the paw until it bent slightly. The force applied to the ipsilateral hindpaw of the animal to induce 5 responses out of 10 trials was recorded as paw withdrawal threshold (PWT, g) and expressed as mean±S.E.M. Data were subjected to a two-way Repeated measures ANOVA with ‘treatment’ as a between subject's effect and ‘day’ as a within subject's effect (Dixon W J. Ann Rev Pharmacol Toxicol. 1980 20, 441-62). Post-hoc analyse were conducted using Planned pair-wise comparison (In VivoStat., Clark et al., 2012 J Psychopharmacology 26(8) 1136-1142).

### Complete Freunds Adjuvant (CFA)-Induced Inflammatory Model of Chronic Pain

Intraplantar injection of Complete Freunds adjuvant (CFA) causes an inflammatory reaction which induces hypersensitivity and oedema and mimics some aspects of clinical inflammatory pain. These effects can be investigated using equipment to measure the paw withdrawal threshold. Naive mice distribute their body weight equally between the two hind paws. When the injected hind paw is inflamed and/or painful, the weight is re-distributed so that less weight is put on the affected paw (decrease in weight bearing on injured paw). Weight bearing through each hind limb is measured using an incapacitance tester (Linton Instruments, UK).

Control group: PBS, 10 mL/kg.

Reference group: Indomethacin 10 mg/kg p.o.

Animals: Male, Naive C57Bl6 mice were acclimatised to the procedure room in their home cages, with food and water available ad libitum.

Environmental conditions: Animals were housed in standard caging and laboratory conditions in groups of 2-5, with free access to food (5CR4, Purina) and water (except during placement in the incapacitance box) on a 12/12 light/dark cycle. Habituation to the incapacitance tester was performed over several days. Weight bearing: Mice were placed in the incapacitance tester with the hind paws on separate sensors and the average force exerted by both hind limbs was recorded over 2 seconds. Baseline weight bearing recordings were taken prior to induction of insult. Inflammatory hypersensitivity was induced by intraplantar injection of CFA (20 μl of 1.5 mg/ml solution) into the left hind paw. Prior to test compound treatment, weight bearing readings were taken to assess baseline hypersensitivity 23 hours post-CFA. Animals were then ranked and randomised to treatment groups according to the CFA response window in a Latin square design. 24 hours post CFA animals were treated with test compound (typically 0.1, 0.3, 1 & 3 mg/kg i.p.), vehicle (PBS) or Indomethacin (10 mg/kg p.o.) All treatments were normalised to a 10 ml/kg dose volume. Weight bearing was then assessed at 4, 24, 48 and 72 hours post-test compound treatment.

Weight bearing (g) readings were taken for both right and left hind paws and the difference calculated. Data were expressed as % ratio ipsilateral/contralateral (mean±S.E.M.) Data were statically integrated by Repeated measures ANOVA followed by Planned comparison test using InVivoStat (invivostat.co.uk, Clark et. al., 2012).

### Chemotherapy-Induced Model of Chronic Pain

Oxaliplatin, a platinum-based chemotherapy drug, is commonly used for the treatment of various types of cancer, including colorectal (Ohsawa et al. J Pharmacol Sci, 2014, 125(3):292-9). The clinical value of Oxaliplatin however, is reduced by acute and chronic forms of peripheral neuropathy including mechanical hyperalgesia that appear as side-effects in both humans (Pasetto et al. Rev Oncol Hematol, 2006 August; 59(2):159-68) and rodents (Ling et al., Toxicology, 2007 May; 234(3):176-84). Oxaliplatin-induced peripheral neuropathy is the most frequent dose-limiting toxicity associated with the therapy, and currently no treatment for it is available (references in Ling et al., 2007).

In rodents a single intraperitoneal injection of Oxaliplatin induces a long-lasting cold hypersensitivity (e.g. Ling et al. Pain, 2007 April; 128(3):225-34, Zhao et al. Mol Pain, 2012 July 28; 8:55) and can be used to model both thermal and mechanical hyperalgesia mimicking aspects of clinical Oxaliplatin-induced neuropathy (Descoeur et al., EMBO Mol Med. 2011 May; 3(5):266-78).

Control group: PBS, 10 mL/kg.

Reference group: Pregabalin (15 mg/kg p.o.)

Animals: Male, Naive C57Bl6 mice were age 6 weeks on arrival and were acclimatised to the procedure room in their home cages, with food and water available ad libitum.

Environmental conditions: Animals were housed in standard caging and laboratory conditions in groups of 2-4, with free access to food (5CR4, Purina) and water (except during placement in the test box) on a 12/12 light/dark cycle. Environmental enrichment was supplied from arrival day and changed every three days to help prevent autotomy.

Oxaliplatin-induced hypersensitivity: A single dose of Oxaliplatin was injected intraperitoneally (i.p.) at 10 mg/kg at a volume of 10 ml/kg. Oxaliplatin was dissolved in 5% dextrose to 1 mg/ml before use. Animals were injected in the same order in which they were tested.

Static mechanical tactile allodynia: Static mechanical (tactile) allodynia was assessed by measurement of withdrawal threshold using calibrated (force; g) von-Frey monofilaments (Touch-Test Sensory Evaluator; Scientific Marketing Associates) applied to the plantar surface of the hind paw. Animals were placed in individual Perspex boxes on a raised metal mesh for 30-40 min before the test. A series of graduated von Frey hairs (0.07, 0.16, 0.4, 0.6 and 1 g) was applied in sequence with a protocol of 1 sec on 1 sec off repeated 10 times. Each hair was applied perpendicularly to the centre of the ventral surface of the paw until it bent slightly. The force applied to the hind-paw of the animal to induce 5 responses out of 10 trials was recorded as paw withdrawal threshold (PWT).

Baseline PWT was assessed on three consecutive days (Day-3, Day-2 and Day-1). The mean of Day-2 and Day-1 were considered the baseline prior to Oxaliplatin dosing. Day 0 was the day of Oxaliplatin injection.

Mechanical allodynia was re-assessed on Day 3 and 4 post-administration of a single dose of Oxaliplatin to monitor the development of allodynia. Animals were then ranked and randomised (based on a Latin square design) to treatment groups according to the percentage change (compared to pre-Oxaliplatin baseline) of the mean PWT observed on Days 3 and 4. Only those animals with a PWT of ≥50% change compared with the pre-oxaliplatin baseline were included in the study. Mechanical withdrawal threshold was taken as the primary end-point.

Animals were injected i.p. on Day 5 post Oxaliplatin with FC2-13, vehicle/PBS (10 ml/kg) or Pregabalin (15 mg/kg p.o).

Mechanical allodynia was re-assessed on Day 5 (4 hours post dose) and thereafter determined on Days 6 and 7 after administration of Oxaliplatin (i.e. 24 hours and 48 hours post FC2-13 treatment).

Behavioural assessments were undertaken by an operator who was unaware of the administered test agents or association of any animal to any treatment group to ensure appropriate full blinding of the experiment. Assessment of the effects of Pregabalin took place at 60 mins post-dosing on Day 5.

### Results

Galanin-FC fusions were generated using the human galanin neuropeptide sequence (GenBank: AAB20740.1) and CH2/CH3 regions of human IgG1. Sequences were designed to contain residues of the IgG1 hinge region and a short linker between the galanin sequence (SEQ ID NO: 1, full galanin underline) and the FC portions (residues 49-282). Additionally, a 19 amino acid leader sequence (dotted underline) was inserted to facilitate secretion of protein from cells. This region is subsequently cleaved from the Galanin-FC fusion product.

Various truncations of the galanin peptide were also inserted into the FC fusion construct. A short peptide linker AEAAAKEAAAKA (SEQ ID NO: 6) (italics) was added to the design between the galanin peptide and the FC segment (SEQ ID NO: 5), to produce, for example, a construct having the sequence as set out in SEQ ID NO: 2.

After initial exploration in functional assays all peptide FC-fusions produced consisted of the leader sequence and linker type shown in SEQ ID NO: 2.

In Vitro Pharmacological Profiling of Novel hIgG1 FC-Peptide Fusions

In brief, a panel of functional cell-based microtiter-plate assays were developed using recombinant GalR1 and GalR2 receptors stably expressed in CHO cells. Test compounds were typically assayed as 10-point concentration-response curves using an appropriate range to best define curves. Nominally this equated to 10 serial (~3fold) dilutions with highest concentrations of 10-1 μM as appropriate.

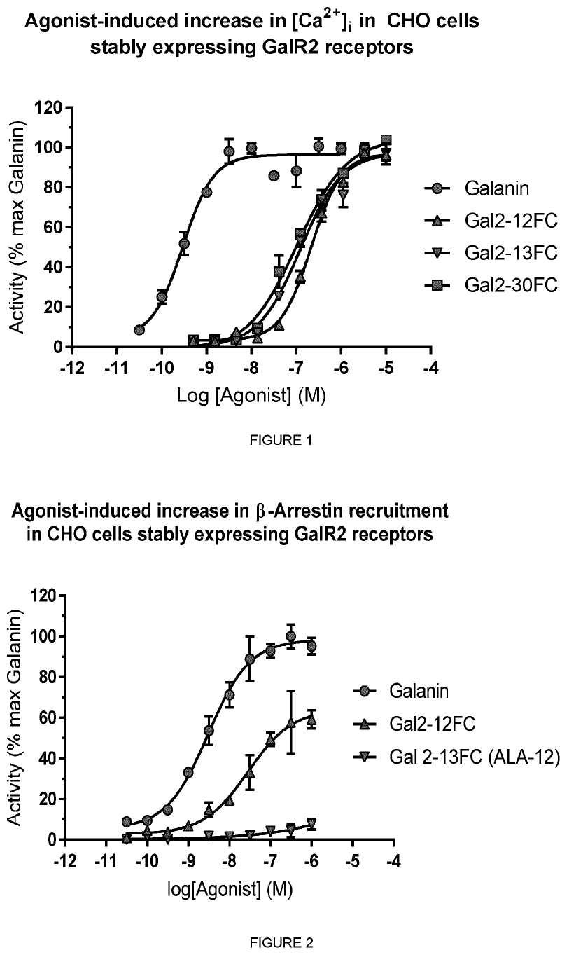

A comprehensive pharmacological characterisation was conducted of both fused and non-fused variants of galanin/galanin truncates, alongside other peptides described in the literature as galanin receptor agonists. In addition to generating both potency and efficacy estimates, the latter being expressed relative to native galanin, both G-protein and non-G-Protein signalling pathways were measured. In keeping with the well described secondary signalling messengers of the two galanin receptors, intracellular calcium mobilization was utilised for GalR2 (FIG. 1) and cyclic AMP (CAMP) monitored for GalR1. Additionally, β-Arrestin (BA) recruitment was measured in both receptor systems.

Tables 2 and 3 and FIGS. 1 and 2 show a direct comparison of potency and relative efficacy of different galanin agonists using these common assay platforms.

To account for the fact that observed potency of agonists is system-dependent e.g. can be affected by receptor expression levels and the efficacy of coupling, empirical molar ratio (EPMR) was used where appropriate to highlight receptor selectivity between compounds. These data provided a system-independent measure of activity and enabled comparison of selectivity of GalR2 vs GalR1 between compounds (Tables 2 and 3).

Notwithstanding the impact of curve definition on maximal asymptote, all FC-fusions in the present dataset tended to be full-agonists at GalR2 yet (some) were also partial agonists at GalR1. Some compounds appeared to display functional selectivity in signalling and efficacies tended to be lower (partial) at GalR1 but not GalR2 with respect to BA. Gal2-13FC was found to be GalR2-specific when compared to GalR1, in marked contrast to Gal2-30FC, Gal2-18FC and Gal2-24FC which are all GalR2-preferring over GalR1.

### Ligand Binding of FC Fusion

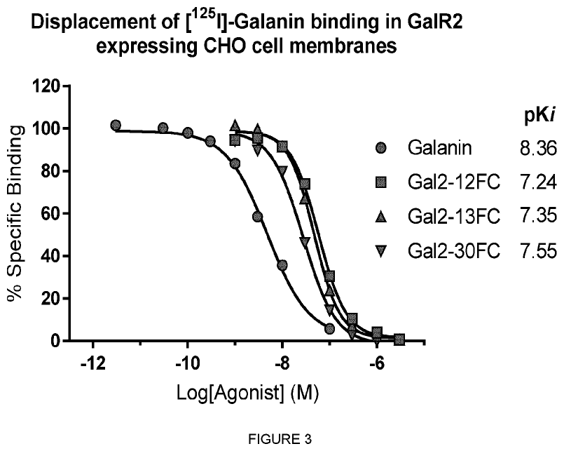

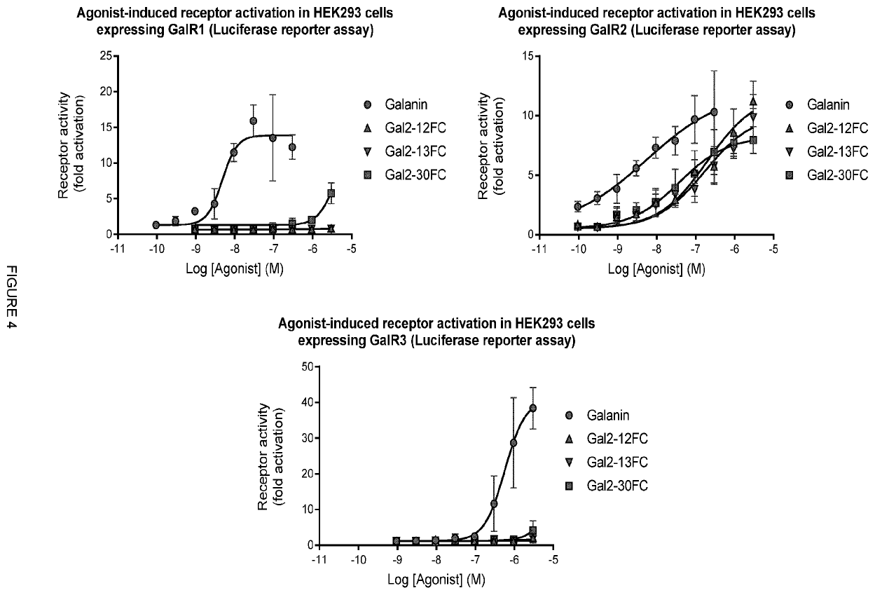

To provide further confidence that responses in functional assays were mediated by galanin receptors and to gauge affinity rather than potency, radioligand binding was conducted on Gal2-12FC, Gal2-13FC Gal2-30FC. Briefly, cell overexpressing recombinant GalR2 were used to for crude membrane preparations. Competition style binding assays were then used to monitor displacement of [125I]-Galanin using a range of concentrations of Gal-FC fusion. After a period of incubation data were expressed relative the maximum specific Galanin bound. Gal2-12FC, Gal2-13FC and Gal2-30FC all displaced [125I]-Galanin binding in a concentration-dependent manner with pIC50s of 7.24, 7.35 and 7.55 respectively (FIG. 3). These data confirmed active target engagement and affinities were in good agreement with potencies from functional studies.

### Effect of ‘Warhead’ Length

To assess the effect on activity at GalR2 and selectivity vs GalR1 FC fusions were produced with varying lengths of galanin warhead linked to the AEAAAKEAAAKA sequence (SEQ ID NO: 6) and the human IgG1 FC region (SEQ ID NO: 5). It is well known that a fragment consisting of galanin resides 2 to 11 is the smallest fragment able to activate the receptor and this fragment displays selectivity vs GalR1. However, when fused to an FC region this chimeric protein showed no measurable activity at GalR2 (Table 2). Chimeric proteins were generated containing residues 2 to 12, 2 to 13, 2 to 14, 2 to 15, 2 to 16, 2 to 17, 2 to 18, 2 to 24 and 2 to 30, these were termed Gal2-12FC, Gal2-13FC, Gal2-14FC, Gal2-15FC, Gal2-16FC, Gal2-17FC, Gal2-18FC, Gal2-24FC and Gal2-30FC. Comparable activity at GalR2 was observed for all FC fusions tested (Table 6), indicating that Gal2-12FC is the smallest fragment of galanin that can be used to generate a GalR2 activating chimeric protein. As the size of the galanin fragment increases the FC-fusions show increased activity at GalR1. (Table 6). Gal2-14FC and above showed increased GalR1 activity compared to Gal2-12FC and Gal2-13FC. These data confirm Gal2-12FC and Gal2-13FC are the most selective chimeric proteins.

### Effect of Linker

To assess the in vitro effect of the polypeptide linker region (residues between the galanin 2-13 fragment and the FC fragment) the AEAAAKEAAAKA sequence (SEQ ID NO: 6) was altered to GGS (SEQ ID NO: 11), GGSGGS (SEQ ID NO: 12), GGSGGSGGSGGS (SEQ ID NO: 13), PAPAPAPA (SEQ ID NO: 14) or a no linker variant in which the galanin fragment is immediately upstream of the fc fragment. These FC fragments were then analysed in vitro for activity at GalR2 or GalR1. The Gal-FC fusion proteins containing GGS linkers showed comparable activity to the fusions containing the AEAAAKEAAAKA (SEQ ID NO: 6) linker (Table 7). The FC fusions containing the PAPAPAPA (SEQ ID NO: 14) linker or ‘no linker’ were more active at GalR2. However, this was also associated with an increase in activity at GalR1, thus offering no net gain in selectivity

### GalR3 Activity

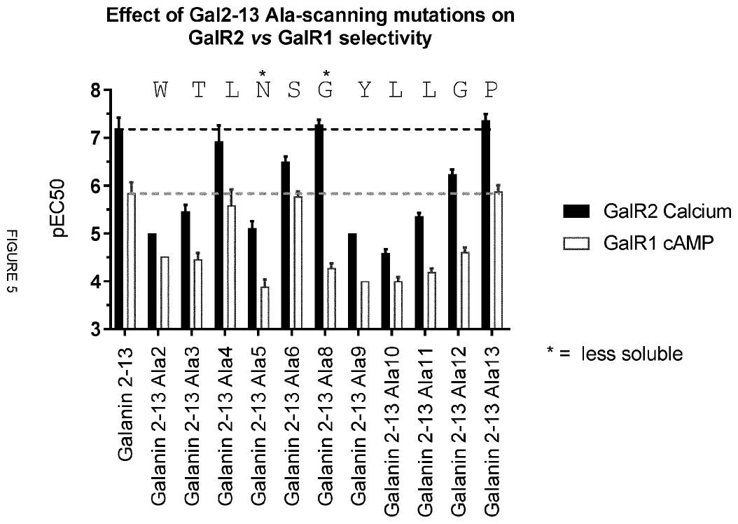

Three galanin receptors have been identified GalR1, GalR2 and GalR3. Whilst GalR1 and GalR2 have been well characterised the exact downstream signalling mechanisms utilised by GalR3 are not fully understood. Therefore, to measure activity at the GalR3 receptor a reporter gene assay was used. In this system MAP/ERK signalling is used to measure receptor activity. Activation of Gqi coupled receptors leads to downstream activation of ERK1/2. The activated ERK translocates to the nucleus where it phosphorylates and activates transcription factors, which lead to the binding of Serum response element (SRE). In this assay SRE activation induces expression of a luciferase reporter gene. Gal2-12FC, Gal2-13FC and Gal2-30FC showed no measurable activity at GalR3 (Table 5). Galanin 1-30, and the Gal2-11 fragment showed expected activity at GalR3. Similarly, Spexin has been described to be a GalR3 specific agonist and this also was able to activate GalR3. To ensure this effect was not an artifact of the reporter gene assay, GalR1 and GalR2 reporter assays (using SRE and NFAT-RE respectively) were also run. Gal2-12FC, Gal2-13FC and Gal2-30FC all showed activity at GalR2 but not GalR1 (FIG. 4). Gal2-11 activated GalR2 but not GalR1 and Galanin 1-30 was active in both (Table 5). These data confirm the Gal fc fusions do not activate GalR3 and are selective for GalR2.

### Orthologue Activity

Gal2-12FC, Gal2-13FC and Gal2-30FC were analysed for activity at GalR2 from species Mus musculus, Rattus rattus and Macaca fascicularis. These species were chosen as they represent potential downstream in vivo models to be used. Within native galanin amino acid residues 1-15 are conserved across species, we would therefore expect the Gal FC fusions comprising amino acids 2 to 13 to retain GalR2 activity in these species. Calcium imaging was used to define receptor activity. For each FC-fusion activity at the orthologue receptors was comparable to that observed at the human receptor (Table 8). These data indicate Gal2-12FC, Gal2-13FC and Gal2-30FC show activity at GalR2 orthologues beyond the human receptor and there is no species specific effect from the FC region.

### Ala Scanning

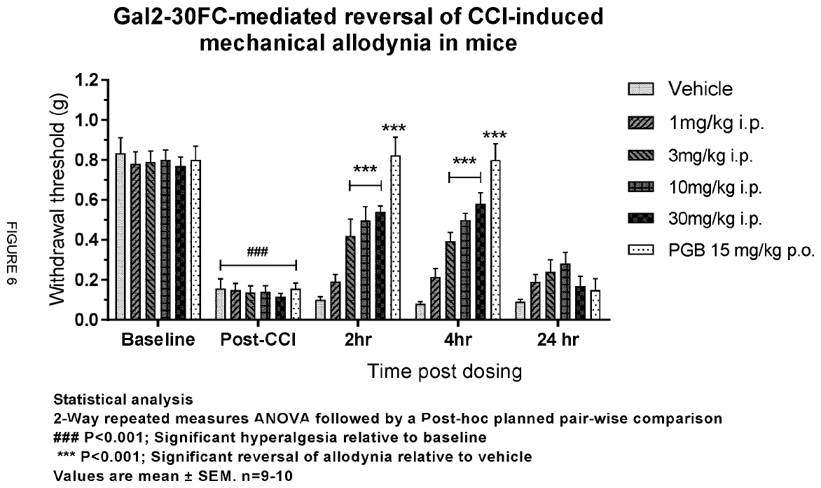

To investigate the role of specific residues within the galanin fragment and to assess whether the potency of Gal2-13FC could be increased 13 reside peptides were produced in which the native amino acid was replace by an alanine reside (‘ala scanning’). The peptides produced are summarised in Table 9. Replacement of residues 2, 3, 5, 6, 9, 10, 11, 12 (corresponding to W, T, L, S, Y, L, L, G) led to a significant decrease in activity at GalR2 (FIG. 5). Replacement of residues 4, 8, 13 (corresponding to N, G, P) caused no decrease in activity. Similarly, Gal2-13ala8 displayed reduced GalR1 activity, offering potential to increase GalR2 over GalR1 selectivity. Of note, none of the substitutions increased GalR2 or GalR1 agonistic activity.

### In-Vivo Profiling of Novel Human FC Fusion Peptides

Following in vitro optimisation of FC fusions, compounds were assayed in an ‘industry standard’ preclinical model of NP. In brief, initially Gal2-30FC and then Gal2-13FC were tested in the mouse Chronic Constriction Injury (CCI) model in which a phenotype analogous to human NP conditions was induced by nerve damage resulting in static mechanical (tactile) allodynia. Compounds capable of reversing this phenotype were thus considered analgesic, at least within the bounds of this particular model. The CCI model of neuropathic pain involves unilateral loose ligation of three ligatures around the left sciatic nerve at mid-thigh level spaced 1 mm apart. This procedure results in the development of hyperalgesia, allodynia and spontaneous pain (ectopic discharges) which can be measured using mechanical and thermal behavioural assessments. This model is believed to mimic some of the symptoms and aetiology of neuropathic pain observed in the clinic^{64; 65}. In the present study, static mechanical (tactile) allodynia was assessed by measurement of withdrawal threshold using calibrated (force; g) von-Frey (vF) monofilaments applied to the plantar surface of the hind paw. The animals were placed in individual perspex boxes on a raised metal mesh for 30-40 min before testing. A series of graduated von Frey hairs (0.07, 0.16, 0.4, 0.6 and 1 g) were applied in sequence with a protocol of 1 sec on 1 sec off repeated 10 times. Each hair was applied perpendicularly to the centre of the ventral surface of the paw until it bent slightly. The force applied to the hind-paw of the animal to induce 5 responses out of 10 trials was recorded as paw withdrawal threshold (PWT).

Typically, baseline PWT was assessed on three consecutive days (Day-2, Day-1 and Day 0) from the ipsilateral paw and mean responses recorded as baseline withdrawal thresholds. On Day 1, neuropathic pain was induced via ligation of the sciatic nerve. Animals were allowed to recover from surgery and vF assessment of mechanical allodynia taken on days 19 and 22. Animals were ranked and randomised (based on a Latin square design) to treatment groups according to the percentage change (compared to pre-surgery baseline) of the mean mechanical withdrawal threshold observed on days 19 and 22. Treatment groups and cohort sizes were designed to enable appropriate powering and statistical analyses e.g. two-way repeated measures ANOVA and Post-hoc analysis using Planned pair-wise comparison.

Gal2-30FC was tested at 1, 3, 10 and 30 mg/kg i.p. and in keeping with study 1, vF assessments made at 2, 4 and 24 hours post-dose.

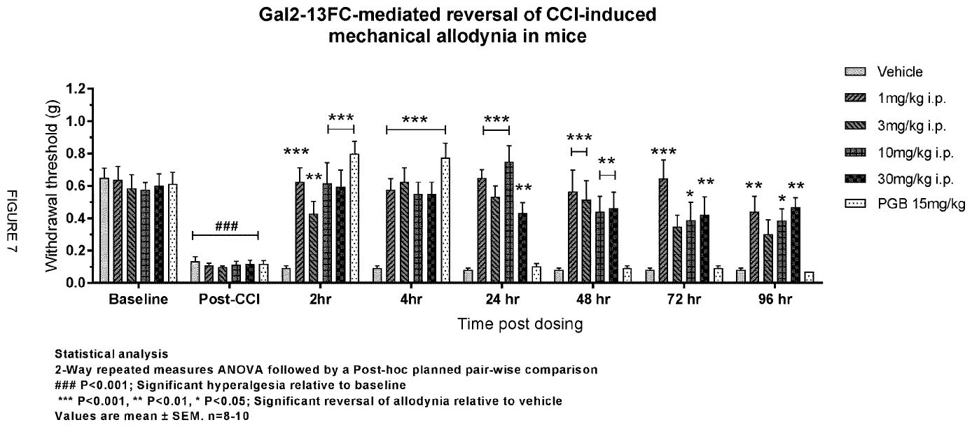

In this study Gal2-30FC was efficacious and reversed CCI-induced mechanical hyperalgesia at 3, 10 and 30 mg/kg i.p. (FIG. 6). Effects were statistically both concentration-dependent and statistically significant. Effects were evident at 2 and 4 hrs but not at 24 hrs, at any of the doses tested. Based on the predicted in vitro stability of human IgG1 FC the lack of effect of Gal2-30FC at 24 hrs was not predicted and suggested that degradation and/or structural change, possibly of the active peptide warhead, was a potential cause.

To explore if shorter galanin fragments within the FC fusion were equally prone to this, as well as testing of molecules more likely to be GalR2 specific over GalR1, additional studies were commissioned. For continuity reasons, study design was kept constant.

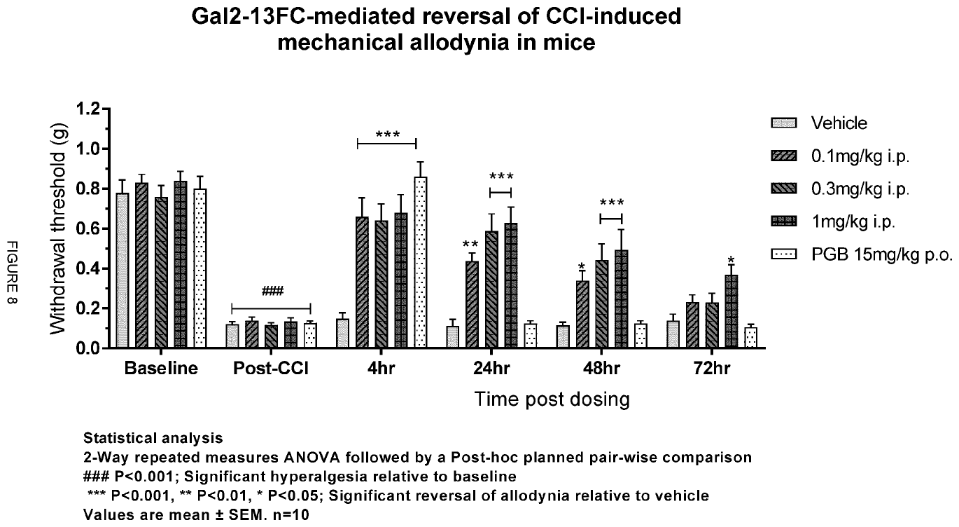

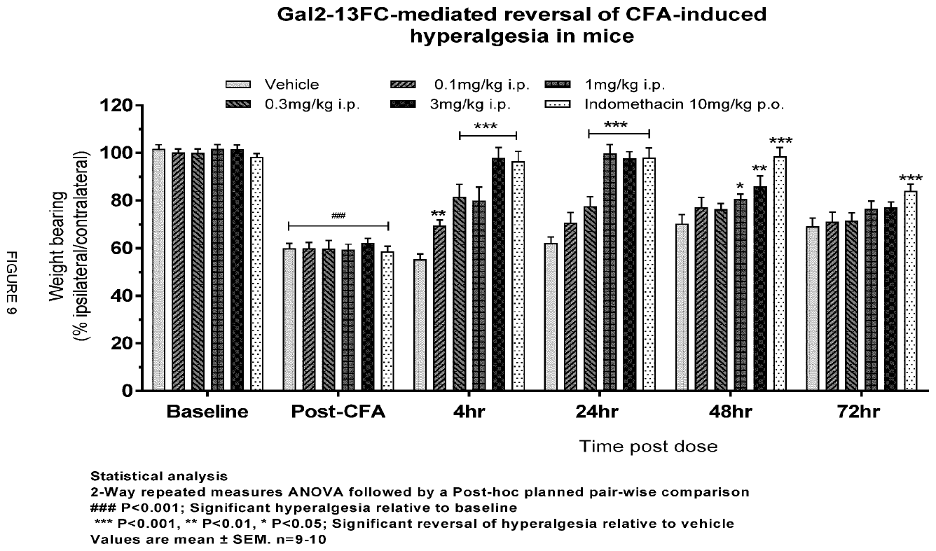

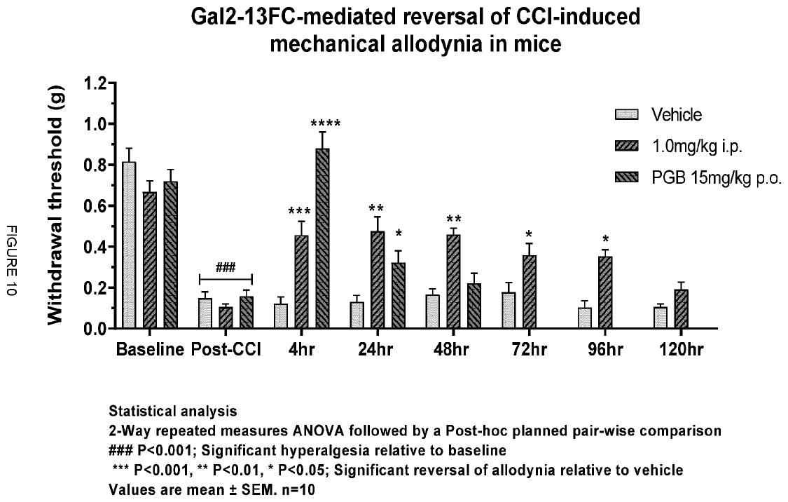

Gal2-13FC was tested at 1, 3, 10 and 30 mg/kg i.p. and vF assessments made at 2, 4 and 24 house post-dose. Gal2-13FC was efficacious and effects statistically significant all doses tested yielding a minimal effective dose of ≤1 mg/kg i.p., (FIGS. 7, 8, and 10). However, in stark contrast with Gal2-30FC, effects were maintained and statistically significant beyond 24 hrs. In this study, the experimenters continued to measure vF for as long as the effect lasted or until was practically possible. Notwithstanding slight and typical variation from cohort to cohort, statistically significant reversal of mechanical hyperalgesia was evident up to 96 hrs post-dose and at both highest and particularly lowest doses tested in these studies (FIGS. 7, 8, and 10).

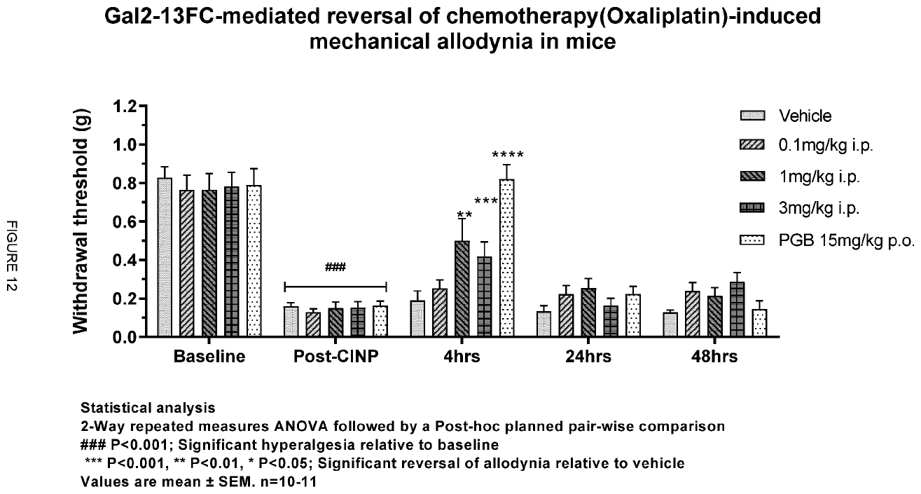

Gal2-13FC was further tested at doses of 0.01, 0.03, and 0.1 mg/kg i.p. and vF assessments made at 4, 24 and 48 hours. This study showed that Gal2-13FC is effective at low doses. A dose of 0.03 mg/kg i.p. was shown to be effective at 4 hours. The effects of a dose of 0.1 mg/kg, which whilst higher is still considered a relatively low dose, were still statistically significant at 24 hours (FIG. 11).

Of note, the difference in minimal effective dose (at 4 hrs) between Gal2-13FC (0.03 mg/kg i.p.) and Gal2-30FC (3 mg/kg i.p.) was not predicted by the in vitro potency which was largely comparable (FIG. 1 and FIG. 4). Although not explicitly demonstrated for these molecules, characteristics such as recycling through FCRn and renal excretion were likely to be virtually identical between Gal2-13FC and Gal2-30FC.

To assess the potential for effect-desensitisation, Gal2-13FC was tested 0.3 and 1 mg/kg i.p. (as above) and vF assessments made again after repeat administration, After an initial dose, followed by vF assessments at 4, 24 and 48 hours, animals were re-dosed and reassessed by vF on days 3, 5, 7, 9, 11 and 13. Within the bounds of acceptable experimental variation there was no loss of effect at either dose and for the entire duration of the study (FIG. 13).

Taken collectively, in multiple studies Gal2-13FC showed robust, efficacious (Minimum Effective Dose [MED]≤0.03 mg/kg i.p.), long-lasting (296 hrs at 1 mg/kg i.p.) and, on repeat dosing (≥13 days), sustained reversal of mechanical hyperalgesia induced by CCI in a preclinical model of NP using mice (FIGS. 7, 10, 11 and 13). In particular the Gal2-13 peptide consistently and repeatedly provides a long-lasting effect in vivo. Whilst in vitro studies Gal2-13 was shown to be more stable.

Additionally, analgesia imparted by Gal2-13FC was not associated with overt sedation, at least observationally during testing, which contrasts sharply with, and which greatly limits the utility of, clinically relevant drugs e.g. gabapentin and morphine.

In Gal2-13FC amino acids 12 and 13 were glycine and proline, respectively. In Gal2-12FC the proline was absent, albeit replaced by the first amino of the linker sequence (alanine). To further explore the importance of glycine and proline in these positions, Gal2-13FC (ALA-12) was made where the proline at position 12 had been mutated to an alanine. This compound, despite being very similar to Gal2-13FC e.g. equal in amino acid length and containing the proline at position 13, failed to activate GalR2 in vitro. Taken collectively, these data suggest glycine and proline, in positions 12 and 13, respectively, dramatically affect functional activity, at least when presented as FC fusions. (Table 4).

The experiments above demonstrated the preferred in vivo profile of Gal2-13FC. In so doing, the study also provided firm confirmation of the involvement and therapeutic potential of galanin and related peptides when interacting with peripheral GalR2 receptors in the aetiology of NP. This was also the first positive demonstration of the utility of a biotherapeutic, specifically an FC fusion, for the treatment of NP and appears to obviate the possibility of GalR2 desensitisation. The predicted lack of CNS penetrance of the FC-fusion and the known receptor selectivity of GalR2 over GalR1 imparted by the design of the peptide warhead, also delineated the roles of central and peripheral GalR2 and GalR1 receptors in this phenotype.

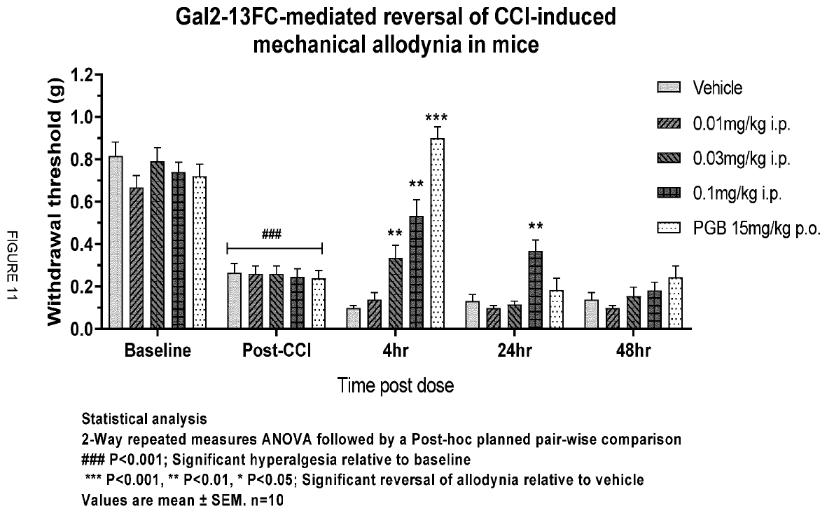

Importantly, Gal2-13FC showed robust, efficacious (MED≤1 mg/kg i.p.) and long lasting reversal (≥72 hrs) of mechanical hyperalgesia induced by CFA in a preclinical mouse model of inflammatory pain mice (FIG. 10). This demonstrates that Gal2-13FC is efficacious in inflammatory pain as well as NP over an extended period at low doses.

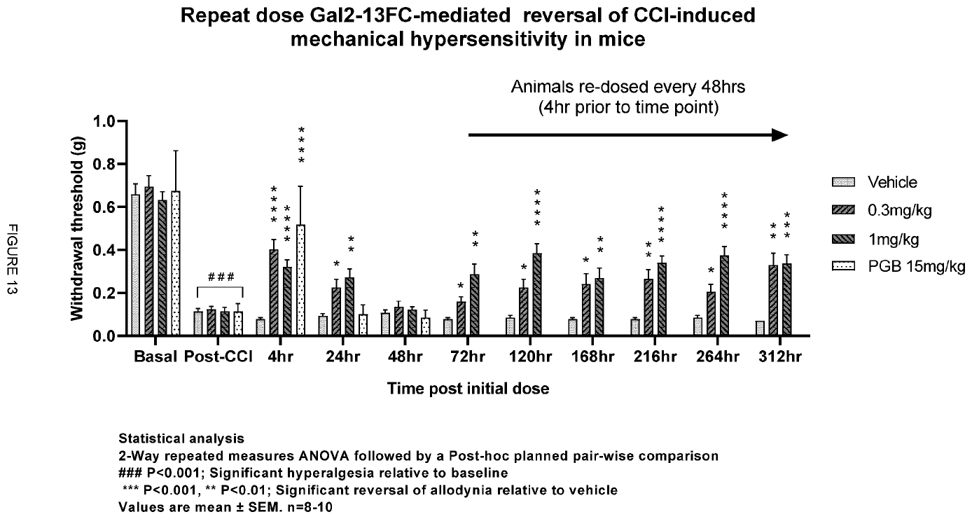

Gal2-13FC was also tested at doses of 0.1, 1 and 3 mg/kg i.p. in a chemotherapy induced model of neuropathic pain and assessments made at 4, 24, and 48 hours post-dose. In this study Gal2-13FC was efficacious and capable of maintaining the reversal of Oxaliplatin-induced mechanical hyperalgesia at 4 hours post dose (day 5 after Oxaliplatin administration) (FIG. 12). This study demonstrates the utility of the Gal2-13FC for the treatment of neuropathic pain associated with anti-cancer therapies.

These data provide clear validation of the role of peripheral activation of GalR2 receptors in nociception and that modulation of the galanin/galanin receptor system affords the prospect of therapeutic intervention capable of circumventing central CNS side-effect liabilities of current front line medication e.g. sedation, constipation, addiction, nausea and so on. Differences in the in vivo profile of Gal2-30FC compared to those of Gal2-13FC would not have been predicted based on their relative activity in vitro (FIG. 3).

Table 1: Galanin agonists; i, full length human galanin (1-30) (SEQ ID NO: 15); ii, full length rat galanin (1-29) (SEQ ID NO: 16); iii, rat galanin truncate 2-29 (SEQ ID NO: 17); iv, galanin truncate 2-11 (SEQ ID NO: 18); v, galanin truncate 1-15 (SEQ ID NO: 19); vi, M617 (SEQ ID NO: 20); vii; M1145 (SEQ ID NO: 21); viii, Nax409-9 (Nax409-9 is PEGylated at the lysine residue) (SEQ ID NO: 22). Number donates amino acid.

TABLE 2
Pharmacology of tested ligands at GalR2.

GalR2

| | Calcium | Beta Arrestin |
|---|---|---|
| Galanin 1-30 (human) | 9.5 +/− 0.4 | 8.3 +/− 0.0 |
| Galanin 2-29 (rat) | 9.2 +/− 0.5 | 8.1 +/− 0.0 |
| Galanin 1-29 (rat) | 9.5 +/− 0.3 | 8.7 +/− 0.1 |
| Galanin 1-15 | 8.6 +/− 0.2 | 7.2 +/− 0.2 |
| Galanin 2-11 | 7.6 +/− 0.2 | 6.4 +/− 0.2 |
| Galnon | 4.7 +/− 0.2 | 3.8 +/− 0.2 |
| M617 | 9.6 +/− 0.4 | 8.4 +/− 0.2 |
| M1145 | 8.9 +/− 0.4 | 7.9 +/− 0.3 |
| Nax409-9 | 6.9 +/− 0.4 | 5.8 +/− 0.5 |
| Gal1-30FC (SEQ ID NO: 23) | 6.4 +/− 0.3 | 5.7 +/− 0.4 |
| Gal2-11FC (SEQ ID NO: 24) | — | — |
| Gal2-12FC (SEQ ID NO: 25) | 6.3 +/− 0.3 | 7.6 +/− 0.2* |
| Gal2-13FC (SEQ ID NO: 7) | 6.3 +/− 0.3 | 6.7 +/− 0.2 |
| Gal2-18FC (SEQ ID NO: 26) | 6.7 +/− 0.1 | 7.3 +/− 0.1 |
| Gal2-24FC (SEQ ID NO: 27) | 6.9 +/− 0.3 | 7.6 +/− 0.0 |
| GAL2-30FC (SEQ ID NO: 28) | 6.6 +/− 0.3 | 7.3 +/− 0.2 |

For all assays n ≥ 3 from three independent experiments.
Data are mean pEC₅₀ +/− standard deviation.
— denotes Inactive.
*denotes not full agonist.
Intrinsic activity 72.1% +/− 9.

TABLE 3
Pharmacology of tested ligands at GalR1.

| | GalR1 | | | |
|---|---|---|---|---|
| | cAMP | | Beta Arrestin | |
| | pEC₅₀ | IA (%) | pEC₅₀ | IA (%) |
| Galanin 1-30 (human) | 9.8 +/− 0.3 | 100.2 +/− 8.5 | 8.97 +/− 0.06 | 97.6 +/− 3.8 |
| Galanin 2-29 (rat) | 8.6 +/− 0.4 | 90.8 +/− 10.5 | 8.09 +/− 0.09 | 67.2 +/− 6.8 |
| Galanin 1-29 (rat) | 9.2 +/− 0.3 | 94.5 +/− 3.4 | 8.90 +/− 0.09 | 95.4 +/− 8.3 |
| Galanin 1-15 | 9.3 +/− 0.2 | 94.4 +/− 17.0 | 8.33 +/− 0.19 | 69.9 +/− 9.2 |
| Galanin 2-11 | N/A | 10.2 +/− 24.1 | N/A | 1.3 +/− 0.5 |
| Galnon | N/A | 2.9 +/− 17.5 | N/A | 40.1 +/− 5.5 |
| M617 | 10.0 +/− 0.5 | 90.1 +/− 10.5 | 8.68 +/− 0.10 | 99.0 +/− 9.9 |
| M1145 | 6.8 +/− 0.4 | 86.7 +/− 14.6 | N/A | 24.5 +/− 2.0 |
| Nax409-9 | 6.5 +/− 0.5 | 66.0 +/− 12.9 | N/A | 7.0 +/− 4.0 |
| Gal1-30FC | 8.4 +/− 0.2 | 93.4 +/− 11.6 | 5.72 +/− 0.40 | 88.1 +/− 7.1 |
| Gal2-11FC | NT | NT | NT | NT |
| Gal2-12FC | NT | NT | NT | NT |
| Gal2-13FC | N/A | 7.5 +/− 15.3 | N/A | 0.8 +/− 1.9 |
| Gal2-18FC | 6.3 +/− 0.3 | 59.8 +/− 15.7 | N/A | 27.3 +/− 5.2 |
| Gal2-24FC | 6.5 +/− 0.3 | 65.8 +/− 10.7 | N/A | 44.9 +/− 8.4 |
| GAL2-30FC | 7.0 +/− 0.4 | 80.2 +/− 14.8 | N/A | 42.8 +/− 8.5 |

For all assays n ≥ 3 from three independent experiments.
Data are mean +/− standard deviation.
Note;
Intrinsic Activity (IA) denotes maximum response obtained at the highest concentration tested (1 μM, except galnon at 100 μM), relative to galanin.
N/A denotes activity failed to reach 50% of that of galanin.

TABLE 5
Activity of Galanin and Gal-FC fusions
at GalR1, R2 and R3 receptors.

| | GalR1 | GalR2 | | GalR3 | |
|---|---|---|---|---|---|
| Galanin 1-30 (human) | 4.1 nM | 5.0 | nM | 570.4 | nM |
| Gal2-11 | — | 36.8 | nM | 1204 | nM |

| | | | | |
|---|---|---|---|---|
| Gal2-12FC | — | 320.3 | nM | — |
| Gal2-13FC | — | 311.5 | nM | — |
| Gal2-30FC | * | 59.0 | nM | — |

| | | | | |
|---|---|---|---|---|
| Spexin | n.d. | n.d. | 9.1 | nM |

All data n ≥ 3.
N.d = not determined,
* denoted only activity at top concentration (1 μM)

TABLE 6
Activity of Gal-FC fusions of different peptide warhead
length at GalR2 and GalR1. GalR2 activity measured using
calcium assay, GalR1 activity measured using cAMP assay.

| | GalR2 | GalR1 |
|---|---|---|
| Galanin 1-30 (human) | 9.5 +/− 0.4 | 9.8 +/− 0.3 |
| Gal2-12FC (SEQ ID NO: 25) | 6.6 +/− 0.0 | 5.4 +/− 0.1 |
| Gal2-13FC (SEQ ID NO: 7) | 6.4 +/− 0.1 | 5.3 +/− 0.2 |
| Gal2-14FC (SEQ ID NO: 29) | 6.7 +/− 0.1 | 5.8 +/− 0.3 |
| Gal2-15FC (SEQ ID NO: 30) | 6.8 +/− 0.1 | 6.5 +/− 0.3 |
| Gal2-16FC (SEQ ID NO: 31) | 6.9 +/− 0.1 | 6.6 +/− 0.4 |
| Gal2-17FC (SEQ ID NO: 32) | 6.6 +/− 0.1 | 6.9 +/− 0.4 |
| Gal2-18FC (SEQ ID NO: 26) | 6.7 +/− 0.1 | 6.3 +/− 0.3 |
| Gal2-24FC (SEQ ID NO: 27) | 6.9 +/− 0.3 | 6.5 +/− 0.3 |
| Gal2-30FC (SEQ ID NO: 28) | 6.8 +/− 0.1 | 7.0 +/− 0.4 |

For all assays n ≥ 3 from three independent experiments.
Data are mean +/− standard deviation.
Highest concentration tested for each 10 μM

TABLE 7
Activity of Gal-FC fusions of different peptide linker length
at GalR2 and GalR1.GalR2 activity measured using calcium
assay, GalR1 activity measured using cAMP assay.

| | GalR2 | GalR1 |
|---|---|---|
| Gal2-13FC (SEQ ID NO: 7) | 6.4 +/− 0.1 | 5.1 +/− 0.2 |
| Gal2-13FC GGS (SEQ ID NO: 33) | 6.4 +/− 0.1 | 5.3 +/− 0.1 |
| Gal2-13FC GGS2 (SEQ ID NO: 34) | 6.4 +/− 0.0 | 5.5 +/− 0.2 |
| Gal2-13FC GGS4 (SEQ ID NO: 35) | 6.2 +/− 0.1 | 5.3 +/− 0.0 |
| Gal2-13FC NL (SEQ ID NO: 36) | 7.3 +/− 0.1 | 5.9 +/− 0.2 |
| Gal2-13FC PA4 (SEQ ID NO: 37) | 6.8 +/− 0.1 | 6.1 +/− 0.0 |

GSS (SEQ ID NO: 11), GSS2 (SEQ ID NO: 12), GSS4 (SEQ ID NO: 13) - GSS linkers; NL—No Linker; PA4 - PAPAPAPA linker (SEQ ID NO: 14)
For all assays n ≥ 3 from three independent experiments.
Data are mean +/− standard deviation.
Highest concentration tested for each 10 μM

TABLE 8
Activity of Gal-FC fusions of different peptide length
at mouse GalR2, rat GalR2 and cynomolgus GalR2.

| | Mouse | | Cynomolgus |
|---|---|---|---|
| | GalR2 | Rat GalR2 | GalR2 |
| Galanin 1-30 (human) | 9.0 +/− 0.12 | 8.8 +/− 0.3 | 9.6 +/− 0.2 |
| Gal2-12FC | 6.8 +/− 0.1 | 6.6 +/− 0.4 | 7.7 +/− 0.4 |
| Gal2-13FC | 6.4 +/− 0.3 | 6.5 +/− 0.1 | 7.7 +/− 0.4 |
| Gal2-30FC | 7.0 +/− 0.1 | 6.8 +/− 6.9 | 8.4 +/− 0.1 |

Activity measured using Calcium assays.
For all assays n ≥ 3 from three independent experiments.
Data are mean +/− standard deviation.

Sequences

1 76
MEWSGIFLFLVATATDVHSGWTLNSAGYLLGPHAVGNHRSFSDKNGLTSLEPKSSDKTHTCPPCPAPELLGGPSVF
77 151
LFPPKPKDTLMISRTPEVTCVVVDVSHEDPEVKFNWYVDGVEVHNAKTKPREEQYNSTYRVVSVLTVLHQDWING
152 228
KEYKCKVSNKALPAPIEKTISKAKGQPREPQVYTLPPSREEMTKNQVSLTCLVKGFYPSDIAVEWESNGQPENNYKT
229 282
TPPVLDSDGSFFLYSKLTVDKSRWQQGNVFSCSVMHEALHNHYTQKSLSLSPGK
SEQ ID NO: 1-Amino acid sequence of Gal1-30FC fusion.
1 75
MEWSGIFLFLVATATDVHSWTLNSAGYLLGPHAVGNHRSFSDKNGLTSAEAAAKEAAAKALEPKSSDKTHTCPPC
76 151
PAPELLGGPSVFLFPPKPKDTLMISRTPEVTCVVVDVSHEDPEVKENWYVDGVEVHNAKTKPREEQYNSTYRVVSV
152 228
LTVLHQDWLNGKEYKCKVSNKALPAPIEKTISKAKGQPREPQVYTLPPSREEMTKNQVSLTCLVKGFYPSDIAVEWE
229 293
SNGQPENNYKTTPPVLDSDGSFFLYSKLTVDKSRWQQGNVFSCSVMHEALHNHYTQKSLSLSPGK
SEQ ID NO: 2-Amino acid sequence of Gal2-30FC fusion.
MEWSGIFLFLVATATDVHS
SEQ ID NO: 3-Leader sequence
WTLNSAGYLLGP
SEQ ID NO: 4-GalR2 binding moiety Gal2-13
LEPKSSDKTHTCPPCPAPELLGGPSVFLFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSCSVMHEALHNHYTQKSLSLSP
GK
SEQ ID NO: 5-IgG1 FC region
AEAAAKEAAAKA
SEQ ID NO: 6-Linker
WTLNSAGYLLGPAEAAAKEAAAKALEPKSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
SEQ ID NO: 7-Gal2-13FC Mature chimeric protein
(binding moiety underlined, linker italicised)
MEWSGIFLFLVATATDVHSWTLNSAGYLLGPAEAAAKEAAAKALEPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 8-Gal2-13FC Precursor chimeric protein (leader dotted underline,
binding moiety solid underline, linker italicised)
WTX₁NSA X₂YLLGX₃, wherein X₁, X₂ and X₃ are independently any amino acid.
SEQ ID NO: 9
WTX₁NSA X₂YLLGX₃, X₁ is independently L or A, X₂ is independently G or A
and X₃ is independently P or A.
SEQ ID NO: 10
GGS
SEQ ID NO: 11-Linker
GGSGGS
SEQ ID NO: 12-Linker
GGSGGSGGSGGS
SEQ ID NO: 13-Linker
PAPAPAPA
SEQ ID NO: 14-Linker
GWTLNSAGYLLGPHAVGNHRSFSDKNGLTS
SEQ ID NO: 15-full length human galanin (1-30)
GWTLNSAGYLLGPHAI DNHRS FHDKYGLA
SEQ ID NO: 16-full length rat galanin (1-29)
WTLNSAGYLLGPHAI DNHRS FHDKYGLA
SEQ ID NO: 17-rat galanin truncate 2-29
WTLNSAGYLL
SEQ ID NO: 18-Gal 2-11
GWTLNSAGYLLGPHA
SEQ ID NO: 19-Gal 1-15
GWTLNSAGYLLGPQPPGFSPFR
SEQ ID NO: 20-M617
RGRGNWTLNSAGYLLGPVLPPPALALA
SEQ ID NO: 21-M1145
WTLNS AGYLLGPKKKKK
SEQ ID NO: 22-Nax409-9
GWTLNSAGYLLGPHAVGNHRSFSDKNGLTSAEAAAKEAAAKALEPKSSDKTHTCPPCTRUEPAPELLGGPSVELFPP
KPKDTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 23-Gal1-30FC Mature chimeric protein
(binding moiety underlined, linker italicised)
WTLNSAGYLLAEAAAKEAAAKALEPKSSDKTHTCPPCTRUEPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 24-Gal2-11FC Mature chimeric protein
(binding moiety underlined, linker italicised)
WTLNSAGYLLGAEAAAKEAAAKALEPKSSDKTHTCPPCTRUEPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 25-Gal2-12FC Mature chimeric protein
(binding moiety underlined, linker italicised)
WTLNSAGYLLGPHAVGNAEAAAKEAAAKALEPKSSDKTHTCPPCTRUEPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK
SEQ ID NO: 26-Gal2-18FC Mature chimeric protein
(binding moiety underlined, linker italicised)
WTLNSAGYLLGPHAVGNHRSFSDAEAAAKEAAAKALEPKSSDKTHTCPPCTRUEPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 27-Gal2-24FC Mature chimeric protein
(binding moiety underlined, linker italicised)
WTLNSAGYLLGPHAVGNHRSFSDKNGLTSAEAAAKEAAAKALEPKSSDKTHTCPPCTRUEPAPELLGGPSVFLFPPK
PKDTLMISRTPEVTCVVVDVSHEDPEVKENWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 28-Gal2-30FC Mature chimeric protein
(binding moiety underlined, linker italicised)
WTLNSAGYLLGPHAEAAAKEAAAKALEPKSSDKTHTCPPCTRUEPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
SEQ ID NO: 29-Gal2-14FC Mature chimeric protein
(binding moiety underlined, linker italicised)
WTLNSAGYLLGPHAAEAAAKEAAAKALEPKSSDKTHTCPPCTRUEPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
SEQ ID NO: 30-Gal2-15FC Mature chimeric protein
(binding moiety underlined, linker italicised)
WTLNSAGYLLGPHAVAEAAAKEAAAKALEPKSSDKTHTCPPCTRUEPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO: 31-Gal2-16FC Mature chimeric protein
(binding moiety underlined, linker italicised)
WTLNSAGYLLGPHAVGAEAAAKEAAAKALEPKSSDKTHTCPPCTRUEPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 32-Gal2-17FC Mature chimeric protein
(binding moiety underlined, linker italicised)
WTLNSAGYLLGPGGSLEPKSSDKTHTCPPCTRUEPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKGOPREPQVY
TLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 33-Gal2-13FC GGS Mature chimeric protein
(binding moiety underlined, linker italicised)
WTLNSAGYLLGPGGSGGSLEPKSSDKTHTCPPCTRUEPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVE
SCSVMHEALHNHYTQKSLSLSPGK
SEQ ID NO: 34-Gal2-13FC GGS2 Mature chimeric protein
(binding moiety underlined, linker italicised)
WTLNSAGYLLGPGGSGGSGGSGGSLEPKSSDKTHTCPPCTRUEPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPG
SEQ ID NO: 35-Gal2-13FC GGS4 Mature chimeric protein
(binding moiety underlined, linker italicised)
WTLNSAGYLLGPLEPKSSDKTHTCPPCTRUEPAPELLGGPSVFLFPPKPKDTLMISRTPEVTCVVVDVSHEDPEVKE
NWYVDGVEVHNAKTKPREEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKSRWQQGNVFSCSVMH
EALHNHYTQKSLSLSPGK
SEQ ID NO: 36-Gal2-13FC NL Mature chimeric protein
(binding moiety underlined, linker italicised)
WTLNSAGYLLGPPAPAPAPALEPKSSDKTHTCPPCTRUEPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
SEQ ID NO: 37-Gal2-13FC PA4 Mature chimeric protein
(binding moiety underlined, linker italicised)
ATLNSAGYLLGP
SEQ ID NO: 38-Gal2-13 Ala2
WALNSAGYLLGP
SEQ ID NO: 39-Gal2-13 Ala3
WTANSAGYLLGP
SEQ ID NO: 40-Gal2-13 Ala4
WTLASAGYLLGP
SEQ ID NO: 41-Gal2-13 Ala5
WTLNAAGYLLGP
SEQ ID NO: 42-Gal2-13 Ala6
WTLNSAAYLLGP
SEQ ID NO: 43-Gal2-13 Ala8
WTLNSAGALLGP
SEQ ID NO: 44-Gal2-13 Ala9
WTLNSAGYALGP
SEQ ID NO: 45-Gal2-13 Ala10
WTLNSAGYLAGP
SEQ ID NO: 46-Gal2-13 Ala11
WTLNSAGYLLAP
SEQ ID NO: 47-Gal2-13 Ala12
WTLNSAGYLLGA
SEQ ID NO: 48-Gal2-13 Ala13
WTLNSAGYLLGP
SEQ ID NO: 49-GalR2 binding moiety Gal2-12
MRGSHHHHHH
SEQ ID NO: 50
DYKDDDDK
SEQ ID NO: 51
KETAAAKFERQHMDS
SEQ ID NO: 52
WSHPQFEK
SEQ ID NO: 53
EQKLISEEDL
SEQ ID NO: 54
MKAEFRRQESDR
SEQ ID NO: 55
MRDALDRLDRLA
SEQ ID NO: 56
WTLNSAGYLLGPHAVGNHRSFSDKNGLTS
SEQ ID NO: 57-human truncated galanin 2-30
WTLNSAGYLLGPHA
SEQ ID NO: 58-Gal2-15
WTLNSAGYLLGPH
SEQ ID NO: 59-Gal2-14
MEWSGIFLFLVATATDVHSWTLNSAGYLLGAEAAAKEAAAKALEPKSSDKTHTCPPCTRUEPAPELLGGPSVFLFPP
KPKDTLMISRTPEVTCVVVDVSHEDPEVKENWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
SEQ ID NO: 60-Gal2-12FC Precursor chimeric protein
(leader dotted underline, binding moiety solid underline, linker italicised)
WTLNSAGYLLAPAEAAAKEAAAKALEPKSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
SEQ ID NO: 61-Gal2-13FC (Ala12) Mature chimeric protein
(binding moiety underlined, linker italicised)

### REFERENCES

1. Tatemoto, K., et al FEBS Lett. 164, 124-128. 1983.
2. Vrontakis, M. E., et al. Journal of Biological Chemistry. 262, 16755-16758. 1987.
3. Hokfelt, T., et al Neurosci.Lett. 83, 217-220. 1987.
4. Landry, M. et al. Neuroscience. 117, 795-809. 2003.
5. Zhang, X., et al J.Neurocytol. 22, 342-381. 1993.
6. Skofitsch, G. et al Brain Res.Bull. 15, 191-195. 1985.
7. Sun, Y. G. et al Journal Of Neuroscience Research. 85, 2400-2406. 2007.
8. Gray, T. S. et al Neurosci.Lett. 83, 264-268. 1987.
9. Ma, W. and Bisby, M. A. Neuroscience. 79, 1183-1195. 1997.
10. Ma, W. and Bisby, M. A. Neuroscience Letters. 262, 195-198. 1999.
11. Coronel, M. F., et al. Journal of Chemical Neuroanatomy. 35, 94-100. 2008.
12. Villar, M. J. et al Neuroscience. 33, 587-604. 1989.
13. Villar, M. J. et al Exp.Neurol. 112, 29-39. 1991.
14. Shadiack, A. M. et al. Neuropeptides. 32, 257-264. 1998.
15. Reeve, A. J. et al Neurosci.Lett. 295, 25-28. 2000.
16. Wiesenfeld-Hallin, Z. et al Exp.Brain Res. 71, 663-666. 1988.
17. Wiesenfeld-Hallin, Z. et al Neurosci.Lett. 105, 149-154. 1989.
18. Hao, J. X et al. Eur.J.Neurosci. 11, 427-432. 1999.
19. Xu, X. J et al. Neuropeptides. 34, 137-147. 2000.
20. Bacon, A. et al. Neuroreport. 13, 2129-2132. 2002.
21. Holmes, F. E et al. Proc Natl Acad Sci USA. 100, 6180-6185. 2003.
22. Hygge-Blakeman, K. et al Brain Research. 1025, 152-158. 2004.
23. Pope, R. J. et al. Mol. Pain. 6, 1-5. 2010.
24. Habert-Ortoli, E et al. Proc.Natl.Acad.Sci.U.S.A. 91, 9780-9783. 1994.
25. Smith, K. E. et al J.Biol. Chem. 273, 23321-23326. 1998.
26. Wittau, N. et al. Oncogene. 19, 4199-4209. 2000.
27. Kerekes, N et al European Journal of Neuroscience. 18 (11): 2957-66. 2003.
28. Sten Shi, T. J. et al Neurosci.Lett. 237, 57-60. 1997.
29. Xu, Z. Q et al. Neuroreport. 8, 237-242. 1996.
30. Brumovsky, P., et al Brain Research. 1085, 111-120. 2006.
31. Hobson, S. A . . . . J.Neurochem. 99, 1000-1010. 2006.
32. Waters, S. M. et al Neuroscience. 95, 265-271. 2000.
33. Mennicken, F. et al J. Chem. Neuroanat. 24, 257-268. 2002.
34. Blakeman, K. H. et al Neuroscience. 117, 221-227. 2003.
35. Malkmus, S. et al Neuropeptides. 39, 217-221. 2005.
36. Liu, H. X et al Proc Natl Acad Sci USA. 98, 9960-9964. 2001.
37. Lu, X et al Neuropeptides. 39, 165-167. 2005.
38. Robertson, C. R. et al Journal of Medicinal Chemistry. 2010.
39. Runesson, J. et al Neuropeptides. 43, 187-192. 2009.
40. Holst, J. J. et al Diabetologia. 36, 653-657. 1993.
41. Harling, H. et al Am.J.Physiol. 262, E52-E57. 1992.
42. Alier, K. A et al. Pain. 137, 138-146. 2008.
43. Lawrence, J. M., et al. IBRO Abstracts. Melbourne, Australia. 2007.
44. Hulse, R., et al Mol.Pain. 7, 26. 2011.
45. Lipp, E. Genetic engineering and biotechnology news. 28, 1-4. 2008.
46. Drews, J. Science. 287, 1960-1964. 2000.
47. DiMasi, J. A. et al. Journal of Health Economics. 22, 151-185. 2003.
48. Fathi Z, et al Molecular brain research. 1998; 58(1-2):156-69.
49. Smith K E, et al. The Journal of biological chemistry. 1997; 272(39):24612-6.
50. Hedlund P B, et al. Brain research. 1994; 634(1):163-7.
51. Borroto-Escuela D O et al. Biochemical and biophysical research communications. 2014; 452(3):347-53.
52. Land T, et al International journal of peptide and protein research. 1991; 38(3):267-72.
53. Crawley J N, et al The Journal of neuroscience: the official journal of the Society for Neuroscience. 1990; 10(11):3695-700.
54. Land T, L et al Brain research. 1991; 558(2):245-50.
55. Wang S, et al The Journal of biological chemistry. 1997; 272(51):31949-52.
56. Liu H X, et al PNAS USA 2001; 98(17):9960-4.
57. Lang R et al. Pharmacology & therapeutics. 2007; 115(2):177-207.
58. Lundstrom L, et al t. Int J Pept Res Ther. 2005; 11(1):17-27.
59. Robertson C R et al. J Med Chem. 2010; 53(4):1871-5.
60. Metcalf C S et al. The Journal of pharmacology and experimental therapeutics. 2015; 352(1):185-93.
61. Sollenberg U, et al. Neuropeptides. 2005; 39(3):161-3.
62. Bartfai T, et al PNAS USA. 2004; 101(28):10470-5.
63. Scmidt S R. Fusion Protein Technologies for Biopharmaceuticals: Applications and Challenges. 2013.
64. Bennett G J et al. Pain. 1988; 33(1):87-107.
65. Field et al. Pain. 1999; 83(2):303-11.
66. Brunner S M et al PNAS 2014; 111(19):7138-7143

1. A chimeric protein comprising;
(i) a galanin fragment consisting of residues 2-13 of galanin (Gal2-13) or 2-12 of galanin (Gal2-12) or a variant thereof, and
(ii) an FC region.

2. A chimeric protein according to claim 1 which is a selective agonist of peripheral GalR2.

3. A chimeric protein according to claim 1 or claim 2 wherein the galanin fragment is at the N terminus and the FC region is at the C terminus of the chimeric protein.

4. A chimeric protein according to any one of the proceeding claims wherein the galanin fragment consists of residues 2-13 of galanin (Gal2-13) or a variant thereof.

5. A chimeric protein according to any one of the preceding claims wherein the galanin fragment consists of the amino acid sequence of SEQ ID NO: 9.

6. A chimeric protein according to any one of the preceding claims wherein the galanin fragment consists of the amino acid sequence of SEQ ID NO: 10.

7. A chimeric protein according to any one of the preceding claims wherein the galanin fragment consists of the amino acid sequence of SEQ ID NO: 4.

8. A chimeric protein according to any one of the preceding claims wherein the FC region is an IgG1 FC region.

9. A chimeric protein according to any one of the preceding claims wherein the FC region comprises an amino acid sequence having at least 90% sequence identity to SEQ ID NO: 5.

10. A chimeric protein according to any one of the preceding claims wherein the FC region comprises the amino acid sequence of SEQ ID NO: 5.

11. A chimeric protein according to any one of the preceding claims wherein the galanin fragment and the FC region are directly connected.

12. A chimeric protein according to any one of claims 1 to 8 wherein the galanin fragment and the FC region are connected through a linker.

13. A chimeric protein according to claim 12 wherein the linker comprises the amino acid sequence of any one of SEQ ID NOS: 6 and 11-14.

14. A chimeric protein according to any one of the preceding claims comprising an amino acid sequence having at least 90% sequence identity to SEQ ID NO: 7.

15. A chimeric protein according to any one of the preceding claims comprising the amino acid sequence of SEQ ID NO: 7.

16. A chimeric protein according to any one claims 1 to 3 wherein the galanin fragment consists of residues 2-12 of galanin (Gal2-12) or a variant thereof.

17. A chimeric protein according to claim 16 wherein the galanin fragment consists of consists of the amino acid sequence of SEQ ID NO: 49.

18. A chimeric protein according to claim 16 or 17 comprising the amino acid sequence of SEQ ID NO: 25.

19. A chimeric protein according to any one of the preceding claims, wherein the chimeric protein displays analgesic activity in vivo for 48 hours or more.

20. A chimeric protein according to claim 19, wherein the chimeric protein reduces paw withdrawal in a CCI murine model or CFA-induced hyperalgesia murine model for 48 hours or more.

21. A nucleic acid encoding a chimeric protein according to any one of the preceding claims.

22. A vector comprising a nucleic acid according to claim 21.

23. A recombinant cell comprising a nucleic acid according to claim 21 or a vector according to claim 22.

24. A pharmaceutical composition comprising a chimeric protein according to any one of claims 1 to 20 and a pharmaceutically acceptable excipient.

25. A method of treatment of pain in an individual comprising administering a chimeric protein according to any one of claims 1 to 20 to an individual in need thereof.

26. A method according to claim 25 wherein the pain is inflammatory or neuropathic pain.

27. A chimeric protein according to any one of claims 1 to 20 for use in a method of treatment of the human or animal body by therapy.

28. A chimeric protein according to any one of claims 1 to 20 for use in a method of treatment of pain.

29. A chimeric protein for use according to claim 28 wherein the pain is inflammatory or neuropathic pain.


SEQUENCE LISTING (61 sequences)

SEQ ID NO: 1 - protein, 282 aa, synthetic construct
MEWSGIFLFLVATATDVHSGWTLNSAGYLLGPHAVGNHRSFSDKNGLTSLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 293 aa, synthetic construct
MEWSGIFLFLVATATDVHSWTLNSAGYLLGPHAVGNHRSFSDKNGLTSAEAAAKEAAAKALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 19 aa, synthetic construct
MEWSGIFLFLVATATDVHS

SEQ ID NO: 4 - protein, 12 aa, synthetic construct
WTLNSAGYLLGP

SEQ ID NO: 5 - protein, 233 aa, synthetic construct
LEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 12 aa, synthetic construct
AEAAAKEAAAKA

SEQ ID NO: 7 - protein, 257 aa, synthetic construct
WTLNSAGYLLGPAEAAAKEAAAKALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 276 aa, synthetic construct
MEWSGIFLFLVATATDVHSWTLNSAGYLLGPAEAAAKEAAAKALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 12 aa, synthetic construct
  source 1..12: note = Wherein X1, X2 and X3 are independently any amino acid.
WTXNSAXYLLGX

SEQ ID NO: 10 - protein, 12 aa, synthetic construct
  source 1..12: note = X1 is independently L or A, X2 is independently G or A and X3 is independently P or A,
WTXNSAXYLLGX

SEQ ID NO: 11 - skipped

SEQ ID NO: 12 - protein, 6 aa, synthetic construct
GGSGGS

SEQ ID NO: 13 - protein, 12 aa, synthetic construct
GGSGGSGGSGGS

SEQ ID NO: 14 - protein, 8 aa, synthetic construct
PAPAPAPA

SEQ ID NO: 15 - protein, 30 aa, Homo sapiens
GWTLNSAGYLLGPHAVGNHRSFSDKNGLTS

SEQ ID NO: 16 - protein, 29 aa, Rattus rattus
GWTLNSAGYLLGPHAIDNHRSFHDKYGLA

SEQ ID NO: 17 - protein, 28 aa, Rattus rattus
WTLNSAGYLLGPHAIDNHRSFHDKYGLA

SEQ ID NO: 18 - protein, 10 aa, synthetic construct
WTLNSAGYLL

SEQ ID NO: 19 - protein, 15 aa, synthetic construct
GWTLNSAGYLLGPHA

SEQ ID NO: 20 - protein, 22 aa, synthetic construct
GWTLNSAGYLLGPQPPGFSPFR

SEQ ID NO: 21 - protein, 27 aa, synthetic construct
RGRGNWTLNSAGYLLGPVLPPPALALA

SEQ ID NO: 22 - protein, 17 aa, synthetic construct
WTLNSAGYLLGPKKKKK

SEQ ID NO: 23 - protein, 279 aa, synthetic construct
GWTLNSAGYLLGPHAVGNHRSFSDKNGLTSAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 259 aa, synthetic construct
WTLNSAGYLLAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 260 aa, synthetic construct
WTLNSAGYLLGAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 266 aa, synthetic construct
WTLNSAGYLLGPHAVGNAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 272 aa, synthetic construct
WTLNSAGYLLGPHAVGNHRSFSDAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 278 aa, synthetic construct
WTLNSAGYLLGPHAVGNHRSFSDKNGLTSAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 262 aa, synthetic construct
WTLNSAGYLLGPHAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 263 aa, synthetic construct
WTLNSAGYLLGPHAAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 264 aa, synthetic construct
WTLNSAGYLLGPHAVAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 265 aa, synthetic construct
WTLNSAGYLLGPHAVGAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 252 aa, synthetic construct
WTLNSAGYLLGPGGSLEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 255 aa, synthetic construct
WTLNSAGYLLGPGGSGGSLEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 260 aa, synthetic construct
WTLNSAGYLLGPGGSGGSGGSGGSLEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 249 aa, synthetic construct
WTLNSAGYLLGPLEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 257 aa, synthetic construct
WTLNSAGYLLGPPAPAPAPALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 12 aa, synthetic construct
ATLNSAGYLLGP

SEQ ID NO: 39 - protein, 12 aa, synthetic construct
WALNSAGYLLGP

SEQ ID NO: 40 - protein, 12 aa, synthetic construct
WTANSAGYLLGP

SEQ ID NO: 41 - protein, 12 aa, synthetic construct
WTLASAGYLLGP

SEQ ID NO: 42 - protein, 12 aa, synthetic construct
WTLNAAGYLLGP

SEQ ID NO: 43 - protein, 12 aa, synthetic construct
WTLNSAAYLLGP

SEQ ID NO: 44 - protein, 12 aa, synthetic construct
WTLNSAGALLGP

SEQ ID NO: 45 - protein, 12 aa, synthetic construct
WTLNSAGYALGP

SEQ ID NO: 46 - protein, 12 aa, synthetic construct
WTLNSAGYLAGP

SEQ ID NO: 47 - protein, 12 aa, synthetic construct
WTLNSAGYLLAP

SEQ ID NO: 48 - protein, 12 aa, synthetic construct
WTLNSAGYLLGA

SEQ ID NO: 49 - protein, 12 aa, synthetic construct
WTLNSAGYLLGP

SEQ ID NO: 50 - protein, 10 aa, synthetic construct
MRGSHHHHHH

SEQ ID NO: 51 - protein, 8 aa, synthetic construct
DYKDDDDK

SEQ ID NO: 52 - protein, 15 aa, synthetic construct
KETAAAKFERQHMDS

SEQ ID NO: 53 - protein, 8 aa, synthetic construct
WSHPQFEK

SEQ ID NO: 54 - protein, 10 aa, synthetic construct
EQKLISEEDL

SEQ ID NO: 55 - protein, 12 aa, synthetic construct
MKAEFRRQESDR

SEQ ID NO: 56 - protein, 12 aa, synthetic construct
MRDALDRLDRLA

SEQ ID NO: 57 - protein, 29 aa, Homo sapiens
WTLNSAGYLLGPHAVGNHRSFSDKNGLTS

SEQ ID NO: 58 - protein, 14 aa, synthetic construct
WTLNSAGYLLGPHA

SEQ ID NO: 59 - protein, 13 aa, synthetic construct
WTLNSAGYLLGPH

SEQ ID NO: 60 - protein, 279 aa, synthetic construct
MEWSGIFLFLVATATDVHSWTLNSAGYLLGAEAAAKEAAAKALEPKSSDKTHTCPPCTRUEPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 257 aa, synthetic construct
WTLNSAGYLLAPAEAAAKEAAAKALEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK